(19) United States
(12) Patent Application Publication
(10) Pub. No.: US 2023/0309788 A1
(43) Pub. Date: Oct. 5, 2023

(54) MEDICAL SERVICE SUPPORT DEVICE, MEDICAL SERVICE SUPPORT METHOD, AND PROGRAM

(71) Applicant: FUJIFILM Corporation, Tokyo (JP)

(72) Inventors: Yosuke Ohashi, Tokyo (JP); Shinichi Komiyama, Tokyo (JP); Chieko Iwanaga, Tokyo (JP); Yuma Hori, Tokyo (JP); Tatsuya Kobayashi, Tokyo (JP)

(73) Assignee: FUJIFILM Corporation, Tokyo (JP)

(21) Appl. No.: 18/331,944

(22) Filed: Jun. 9, 2023

Related U.S. Application Data
(63) Continuation of application No. PCT/JP2021/041774, filed on Nov. 12, 2021.

(30) Foreign Application Priority Data
Dec. 14, 2020 (JP) 2020-206814

(51) Int. Cl. A61B 1/00; G06T 7/00

(52) U.S. Cl. CPC A61B 1/00009; A61B 1/00045; G06T 7/0012; G06T 2207/10068

(57) ABSTRACT

The medical service support device includes a processor and a memory connected to or built into the processor. The processor is configured to, in a case where N is a natural number, derive a required quantity of medical accessory devices based on standard procedure service time, from when a first medical procedure service, which is an N-th medical procedure service performed by an information processing apparatus, is started to when the first medical procedure service is ended, standard standby time, from when the first medical procedure service is ended to when a second medical procedure service, which is an (N+1)-th medical procedure service performed by the information processing apparatus, is started, and standard washing time, from when the first medical procedure service is ended to when washing of the medical accessory device, which is attachably and detachably connected to the information processing apparatus, is ended, and output medical service support information obtained based on the required quantity.

### CROSS-REFERENCE TO RELATED APPLICATIONS

This application is a continuation application of International Application No. PCT/JP2021/041774, filed Nov. 12, 2021, the disclosure of which is incorporated herein by reference in its entirety. Further, this application claims priority under 35 USC 119 from Japanese Patent Application No. 2020-206814 filed Dec. 14, 2020, the disclosure of which is incorporated by reference herein.

### BACKGROUND

### 1. Technical Field

The present invention relates to a medical service support device, a medical service support method, and a program.

### 2. Related Art

WO2016/199545A discloses an endoscopy service support system including: an examination schedule management unit that manages an examination room for performing an endoscopy and an examination schedule of a plurality of endoscopies including estimated examination start time point information and estimated examination end time point information, and examination type information related to an examination content of the endoscopy; a first allocation processing unit that allocates an endoscope to be used from among a plurality of endoscopes for each of the endoscopies managed by the examination schedule management unit; a second allocation processing unit that allocates a washer for washing an endoscope used in each of the endoscopies from among a plurality of washers; and a washing schedule management unit that manages a washer and a washing schedule for the plurality of endoscopes including estimated washing start time point information and estimated washing end time point information.

The endoscopy service support system described in WO2016/199545A further includes: a status information acquisition unit that acquires status information related to an examination status; and a rescheduling processing unit that determines a need to change the examination schedule and/or the washing schedule based on the status information, in which the rescheduling processing unit instructs at least one of the examination schedule management unit or the washing schedule management unit to change an element included in the examination schedule and/or the washing schedule.

### SUMMARY

One embodiment according to the present disclosed technology provides a medical service support device, a medical service support method, and a program capable of supporting efficient performance of an endoscope procedure service.

A medical service support device according to a first aspect of the present disclosed technology comprises: a processor; and a memory that is connected to or built into the processor, in which the processor is configured to: in a case where N is a natural number, derive a required quantity of endoscopes based on standard procedure service time, from when a first endoscope procedure service, which is an N-th endoscope procedure service performed by an information processing apparatus used together with the endoscope, is started to when the first endoscope procedure service is ended, standard standby time, from when the first endoscope procedure service is ended to when a second endoscope procedure service, which is an (N+1)-th endoscope procedure service performed by the information processing apparatus, is started, and standard washing time, from when the first endoscope procedure service is ended to when washing of the endoscope is ended; and output medical service support information obtained based on the required quantity.

A medical service support device according to a second aspect of the present disclosed technology comprises: a processor; and a memory that is connected to or built into the processor, in which the processor is configured to: in a case where N is a natural number, derive a required quantity of medical accessory devices based on standard procedure service time, from when a first medical procedure service, which is an N-th medical procedure service performed by an information processing apparatus, is started to when the first medical procedure service is ended, standard standby time, from when the first medical procedure service is ended to when a second medical procedure service, which is an (N+1)-th medical procedure service performed by the information processing apparatus, is started, and standard washing time, from when the first medical procedure service is ended to when washing of the medical accessory device, which is attachably and detachably connected to the information processing apparatus, is ended; and output medical service support information obtained based on the required quantity.

A medical service support method according to a third aspect of the present disclosed technology comprises: in a case where N is a natural number, deriving a required quantity of endoscopes based on standard procedure service time, from when a first endoscope procedure service, which is an N-th endoscope procedure service performed by an information processing apparatus used together with the endoscope, is started to when the first endoscope procedure service is ended, standard standby time, from when the first endoscope procedure service is ended to when a second endoscope procedure service, which is an (N+1)-th endoscope procedure service performed by the information processing apparatus, is started, and standard washing time, from when the first endoscope procedure service is ended to when washing of the endoscope is ended; and outputting medical service support information obtained based on the required quantity.

A program according to a fourth aspect of the present disclosed technology that causes a computer to execute a process comprises: in a case where N is a natural number, deriving a required quantity of endoscopes based on standard procedure service time, from when a first endoscope procedure service, which is an N-th endoscope procedure service performed by an information processing apparatus used together with the endoscope, is started to when the first endoscope procedure service is ended, standard standby time, from when the first endoscope procedure service is ended to when a second endoscope procedure service, which is an (N+1)-th endoscope procedure service performed by the information processing apparatus, is started, and standard washing time, from when the first endoscope procedure service is ended to when washing of the endoscope is ended; and outputting medical service support information obtained based on the required quantity.

### BRIEF DESCRIPTION OF THE DRAWINGS

Exemplary embodiments of the technology of the disclosure will be described in detail based on the following figures, wherein:

FIG. 1 is a conceptual diagram showing a schematic configuration of a medical service support system;

FIG. 2 is a flowchart showing an example of a flow of an endoscope handling service;

FIG. 3 is a block diagram showing an example of a hardware configuration of an electrical system of an endoscope part management device;

FIG. 4 is a block diagram showing an example of a function of a CPU and a storage content of a storage in an endoscope part management device main body;

FIG. 5 is a conceptual diagram showing an example of a content of an endoscopy management database;

FIG. 6 is a conceptual diagram showing an example of a content of a washer management database;

FIG. 7 is a block diagram showing an example of a hardware configuration of an electrical system of a support management server;

FIG. 8 is a block diagram showing an example of a function of a CPU and a storage content of a storage in the support management server;

FIG. 9 is a block diagram showing an example of a storage content of a management information storage device;

FIG. 10 is a flowchart showing an example of a flow of a management device control process;

FIG. 11 is a flowchart showing an example of a flow of a medical service support process according to an embodiment;

FIG. 12 is a screen view showing an example of the medical service support screen according to the embodiment;

FIG. 13 is a flowchart showing a modification example of a flow of a medical service support process according to the embodiment;

FIG. 14 is a screen view showing a modification example of the medical service support screen according to the embodiment;

FIG. 15 is a block diagram showing an example of a processing content of generating medical service support information, which includes information obtained based on a required endoscope quantity and a current endoscope quantity, and transmitting the medical service support information to the endoscope part management device;

FIG. 16 is a block diagram showing an example of a processing content of generating medical service support information, which includes information obtained based on a required endoscope quantity and a used endoscope quantity, and transmitting the medical service support information to the endoscope part management device; and

FIG. 17 is a block diagram showing an example of a processing content of generating medical service support information, which includes endoscope requiring update specification information, and transmitting the medical service support information to the endoscope part management device.

### DETAILED DESCRIPTION

An example of an embodiment of a medical service support device, a medical service support method, and a program according to the present disclosed technology will be described with reference to the accompanying drawings.

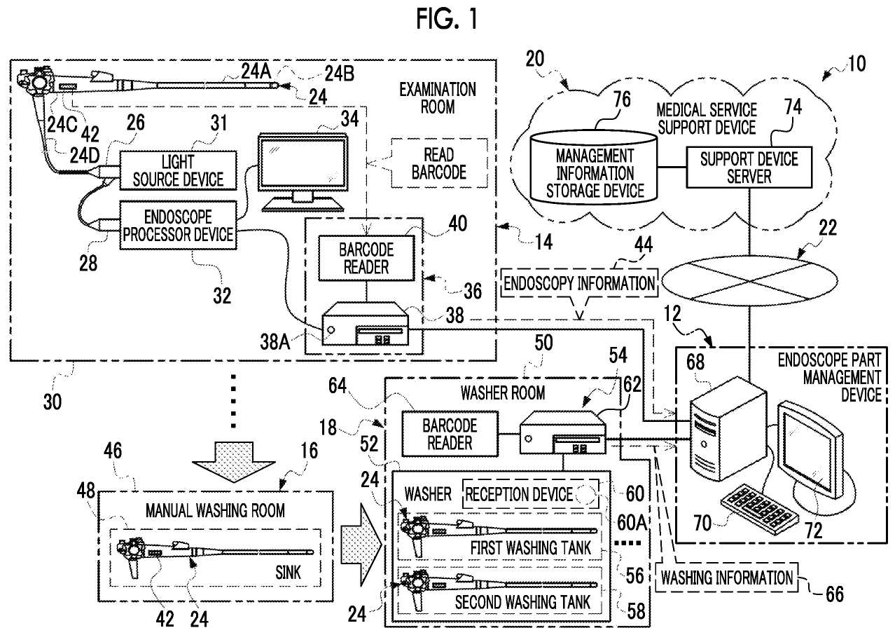

As an example, as shown in FIG. 1, a medical service support system 10 includes an endoscope part management device 12, a plurality of examination room facilities 14, a manual washing facility 16, an automatic washing facility 18, and a medical service support device 20. Here, the medical service support device 20 is an example of a “medical service support device” according to the present disclosed technology.

The medical service support system 10 is a system that supports an endoscope handling service. In the present embodiment, the endoscope handling service refers to, for example, a service performed by a person engaged in examination (hereinafter, also referred to as an “endoscopy worker”) using an endoscope 24 (hereinafter, also referred to as an “endoscopy”) in a medical installation facility (for example, a hospital). The endoscope handling service includes an endoscopy, a manual washing service, and an automatic washing service. The endoscopy is an example of an “endoscope procedure service” according to the present disclosed technology.

The medical installation facility has a plurality of endoscopes 24, and a type of endoscope 24, which corresponds to an examination item, is used in each of the plurality of examination room facilities 14. Examples of the type of endoscope 24 include an upper gastrointestinal scope, a pancreatic duct scope, a bile duct scope, a duodenal scope, a colon scope, an intracerebral scope, an otolaryngology scope, a bronchial scope, and the like.

The endoscope 24 includes an insertion part 24A, an imaging part 24B, an operation part 24C, and a universal cord 24D. The insertion part 24A is inserted into a subject.

The imaging part 24B is provided at a distal end part of the insertion part 24A. The imaging part 24B includes a complementary metal oxide semiconductor (CMOS) type imaging element. A charge-coupled device (CCD) type imaging element may be used instead of the CMOS type imaging element.

The operation part 24C is installed to be continuous with a proximal end side of the insertion part 24A, and a doctor holds the operation part 24C and performs various operations. By operating the operation part 24C, the insertion part 24A is bendable or imaging is performed by the imaging part 24B.

The universal cord 24D is installed to be continuous with the operation part 24C. The universal cord 24D encompasses a light guide (not shown), a fluid tube (not shown), and a signal cable (not shown). Connectors 26 and 28 are provided at an end part of the universal cord 24D. The connector 28 is branched from the connector 26.

A plurality of examination room facilities 14 are provided in the medical installation facility, and each of the examination room facilities 14 includes an examination room 30, a light source device 31, an endoscope processor device 32, a display 34, and an information processing apparatus 36. The light source device 31, the endoscope processor device 32, the display 34, and the information processing apparatus 36 are installed in the examination room 30.

A plurality of information processing apparatuses 36 are present and are provided in each of a plurality of medical sites where the endoscope handling service is performed. In the present embodiment, one information processing apparatus 36 is assigned to each of the examination rooms 30.

The information processing apparatus 36 is an example of an “information processing apparatus” according to the present disclosed technology and is used together with the endoscope 24 in the endoscopy. The information processing apparatus 36 operates every time an endoscopy is performed on one subject in each examination room 30. The information processing apparatus 36 is continuously operated while an endoscopy is performed on one subject. That is, a period from when the operation of the information processing apparatus 36 is started to when the operation of the information processing apparatus 36 is ended corresponds to a period during which an endoscopy is performed on one subject.

The connector 26 is connected to the light source device 31. In a case where the connector 26 is connected to the light source device 31, a light guide and a fluid tube in the universal cord 24D are inserted into the light source device 31. The light source device 31 supplies illumination light, water, and gas to the endoscope 24 via the light guide and the fluid tube. Accordingly, the illumination light is emitted toward a test observation site from an illumination window (not shown) on a distal end surface of the insertion part 24A. Further, water and gas are injected from a nozzle (not shown) on the distal end surface of the insertion part 24A toward the observation window according to the operation performed on the operation part 24C. Here, although an example of the embodiment has been described in which the light source device 31 supplies water and gas to the endoscope 24, this is merely an example, and at least one of water or gas may be supplied to the endoscope 24 from a device other than the light source device.

The connector 28 is connected to the endoscope processor device 32. In a case where the connector 28 is connected to the endoscope processor device 32, a signal cable in the universal cord 24D is electrically connected to the endoscope processor device 32. The endoscope processor device 32 controls the operation of the endoscope 24 by supplying a control signal to the endoscope 24 via a signal cable. Further, the endoscope 24 outputs an imaging signal, which is obtained by causing the imaging part 24B to image the test observation site, to the endoscope processor device 32 via the signal cable.

The endoscope processor device 32 generates a moving image as an image showing the test observation site based on the imaging signal input from the endoscope 24. A frame rate of the moving image is, for example, 30 frames per second (fps). Further, the endoscope processor device 32 generates a still image as an image showing the test observation site in response to an operation performed on the operation part 24C. The endoscope processor device 32 is connected to the display 34 and displays an image, which is generated based on the imaging signal, on the display 34.

In the present embodiment, although the light source device 31 and the endoscope processor device 32 are separate from the information processing apparatus 36, this is merely an example, and at least one of the light source device 31 or the endoscope processor device 32 may be integrated with the information processing apparatus 36.

The information processing apparatus 36 includes an information processing apparatus main body 38 and a barcode reader 40. The information processing apparatus main body 38 is configured to include a computer including a processor and a storage device. Examples of the processor include a central processing unit (CPU). Examples of the storage device include a combination of a non-volatile memory such as an electrically erasable and programmable read only memory (EEPROM), a solid state drive (SSD), and/or a hard disk drive (HDD), and a volatile memory such as a random access memory (RAM).

An examination start button 38A is provided on the information processing apparatus main body 38. The examination start button 38A is an alternate type push button. Further, the examination start button 38A is turned on at a time of starting an endoscopy for one subject and is turned off at a time of ending an endoscopy on one subject. In a case where the examination start button 38A is turned on, the information processing apparatus main body 38 starts the operation, and in a case where the examination start button 38A is turned off, the information processing apparatus main body 38 stops the operation.

The endoscope part management device 12 is connected to the information processing apparatus main body 38. An information processing apparatus ID, which is an identification (ID) capable of specifying the information processing apparatus main body 38, is assigned to the information processing apparatus main body 38, and the endoscope part management device 12 centrally manages each information processing apparatus main body 38, which is installed in each of the examination rooms 30, by using the information processing apparatus ID.

Further, the endoscope processor device 32 is connected to the information processing apparatus main body 38. That is, the endoscope 24 is connected to the information processing apparatus main body 38 via the endoscope processor device 32. Further, a barcode reader 40 is also connected to the information processing apparatus main body 38.

The information processing apparatus main body 38 is operated under the control of the endoscope part management device 12 and controls the operations of the endoscope processor device 32 and the barcode reader 40. The information processing apparatus main body 38 acquires an image, which is generated based on an imaging signal by the endoscope processor device 32, and outputs the acquired image to the endoscope part management device 12.

A barcode 42 is provided on the endoscope 24. In the example shown in FIG. 1, the barcode 42 is provided on the operation part 24C. The barcode 42 is information in which an endoscope ID that is capable of uniquely specifying the endoscope 24 is encrypted. The barcode 42 is read by the barcode reader 40 in a case where an endoscopy is started. The barcode reader 40 outputs the endoscope ID obtained by reading the barcode 42 to the information processing apparatus main body 38.

The barcode reader 40 is also capable of reading a barcode (not shown) that is uniquely assigned to the subject. The barcode, which is assigned to the subject (hereinafter, also referred to as a “subject barcode”), is information in which a subject ID that is capable of uniquely specifying the subject is encrypted, and is attached to, for example, a band (for example, a wristband) attached to the subject's wrist or the like. The subject barcode is read by the barcode reader 40 at a time at which an endoscopy is started.

The information processing apparatus main body 38 outputs various information related to the subject specified through the subject ID and endoscopy information 44 associated with various IDs to the endoscope part management device 12. Here, the various IDs refer to the endoscope IDs obtained by reading the barcode 42 with the barcode reader 40, and the subject IDs obtained by reading the subject barcode with the barcode reader 40. Here, the endoscope IDs and the subject IDs are exemplified as various IDs, but the present disclosed technology is not limited to this, and only the endoscope IDs may be used.

Further, examples of various types of information related to the subject include, for example, an image generated by the endoscope processor device 32, an examination start time point, an examination end time point, and the like. The information processing apparatus main body 38 includes a clock (for example, a real-time clock) and acquires the examination start time point and the examination end time point from the clock. For example, the examination start time point is a time point when the examination start button 38A is turned on, and the examination end time point is a time point when the examination start button 38A is turned off.

The manual washing facility 16 includes a manual washing room 46 and a sink 48. The sink 48 is installed in the manual washing room 46, and manual washing of the endoscope 24 is performed in the sink 48. Here, the manual washing refers to work in which an endoscopy worker manually and physically removes stains on the endoscope 24 by using a detergent, a brush, a sponge, or the like.

The automatic washing facility 18 includes a washer room 50, a plurality of washers 52, and a washer management device 54. The plurality of washers 52 and the washer management devices 54 are installed in the washer room 50. The washer 52 is a device that mechanically washes the endoscope 24 on which manually washing is performed. The plurality of washers 52 include a single-endoscope washer that is capable of washing only one endoscope 24 and a dual-endoscope washer that is capable of washing two endoscopes 24 together. The single-endoscope washer is also conventionally referred to as a single washer, and the dual-endoscope washer is also conventionally referred to as a dual washer.

The single-endoscope washer is provided with only one washing tank into which one endoscope 24 is inserted, whereas the dual-endoscope washer is provided with a first washing tank 56 and a second washing tank 58. One endoscope 24 is inserted into each of the first washing tank 56 and the second washing tank 58. Hereinafter, in a case where it is not necessary to distinguish among the washing tank of the single-endoscope washer, the first washing tank 56, and the second washing tank 58, a term “washing tank” will be used without reference numerals.

The washer 52 includes a reception device 60. The reception device 60 includes a plurality of buttons and a dial. The plurality of buttons include a washing start button 60A. The washing start button 60A is a button that receives an instruction for causing the washer 52 to start washing the endoscope 24 in the washing tank. In a case where the washing start button 60A is turned on, the washer 52 starts the operation. In a case where the washer 52 is operated, the washer 52 sequentially performs washing, rinsing, dehydration, and the like with a detergent for the endoscope 24 in the washing tank according to a default program.

The washing time by the washer 52, that is, the operation time of the washer 52, is designated by operating the dial included in the reception device 60. After a lapse of time designated by operating the dial, the washer 52 stops the operation.

The washer management device 54 includes a washer management device main body 62 and a barcode reader 64. The washer management device main body 62 is configured to include a computer including a processor and a storage device. Examples of the processor include a CPU. Examples of the storage device include a combination of a non-volatile memory such as an EEPROM, an SSD, and/or an HDD, and a volatile memory such as a RAM.

The plurality of washers 52 are connected to the washer management device main body 62, and the washer management device main body 62 centrally manages the plurality of washers 52. For example, the washer management device main body 62 includes a clock and acquires a washing start time point and a washing end time point of each of the plurality of washers 52 from the clock. The washing start time point is a time point when the washing start button 60A of the washer 52 is turned on, and the washing end time point is a time point when the operation of the washer 52 is stopped (for example, a time point when washing of the endoscope 24 in the washing tank is ended).

Here, although an example of the embodiment has been described in which the plurality of washers 52 are centrally managed by the washer management device 54, this is merely an example, and each of a plurality of washer management devices 54 may individually manage the plurality of washers 52, and one washer management device 54 may centrally manage the plurality of washers 52.

Further, here, although a time point when the washing start button 60A is turned on is exemplified as the washing start time point, this is merely an example, and, for example, the washing start time point may be determined according to an instruction given to a device other than the washer management device 54 such as the endoscope part management device 12. Further, here, although a time point when the operation of the washer 52 is stopped is exemplified as the washing end time point, this is merely an example, and the washing end time point may be a time point when a timer for managing the washing time is ended, or a time point when the washing start button 60A is turned off.

A washer ID, which is an ID that is capable of specifying the washer 52, is assigned to each of the plurality of washers 52, and the washer management device main body 62 centrally manages the plurality of washers 52 by using washer IDs.

The barcode reader 64 is connected to the washer management device main body 62. The endoscope part management device 12 is connected to the washer management device main body 62, and the washer management device main body 62 is operated under the control of the endoscope part management device 12 and controls the operation of the plurality of washers 52 and the barcode reader 64.

The barcode 42 of the endoscope 24, on which manual washing is performed, is read by the barcode reader 64. The barcode reader 64 outputs the endoscope ID obtained by reading the barcode 42 to the washer management device main body 62.

The washer management device main body 62 outputs various types of information related to the washer 52 and washing information 66 associated with the endoscope ID corresponding to the endoscope 24 washed by the washer 52 to the endoscope part management device 12. Here, examples of various types of information related to the washer 52 include a washer ID, washing start time point, and washing end time point.

The endoscope 24, the information processing apparatus 36, and the washer 52 are all medical devices used in the endoscope handling service. In the following, for convenience of explanation, in a case where it is not necessary to distinguish among the endoscope 24, the information processing apparatus 36, and the washer 52, a term “medical device” will also be used without reference numerals.

The endoscope part management device 12 is a device that manages a part (endoscope part) that is responsible for the endoscope handling service in a medical installation facility. The endoscope part management device 12 includes an endoscope part management device main body 68, a reception device 70, and a display 72. The display 72 is an example of a “presentation device” according to the present disclosed technology.

As described in detail later, the endoscope part management device main body 68 is a device configured to include a computer. The reception device 70 is connected to the endoscope part management device main body 68. The reception device 70 includes a keyboard, a mouse, a touch panel, and/or the like, and receives an instruction from a user or the like of the endoscope part management device 12. The display 72 is connected to the endoscope part management device main body 68. The display 72 displays various types of information under the control of the endoscope part management device main body 68. Examples of the display 72 include an electro-luminescence (EL) display, a liquid crystal display, and the like.

The endoscope part management device main body 68 acquires the endoscopy information 44 output from the information processing apparatus main body 38 and the washing information 66 output from the washer management device main body 62, and executes a process by using the acquired endoscopy information 44 and washing information 66. In the following, for convenience of explanation, in a case where it is not necessary to distinguish between the endoscopy information 44 and the washing information 66, a term “management target information” will be used without reference numerals.

In the present embodiment, the endoscope part management device 12 is connected to the medical service support device 20 via a network 22. The network 22 is, for example, the Internet. Note that the network 22 is not limited to the Internet and may be a wide area network (WAN) and/or a local area network (LAN) such as an intranet. Further, the endoscope part management device 12 and the medical service support device 20 may be integrally formed, and the medical service support device 20 may have at least some of the functions of the endoscope part management device 12.

The endoscope part management device main body 68 exchanges information with the medical service support device 20 via the network 22. The medical service support device 20 is realized by cloud computing. Here, although cloud computing is exemplified, this is merely an example, and, for example, the medical service support device 20 may be implemented by a mainframe or implemented by network computing such as fog computing, edge computing, or grid computing.

The medical service support device 20 includes a support management server 74 and a management information storage device 76. The management information storage device 76 is connected to the support management server 74.

The support management server 74 is connected to the endoscope part management device main body 68 via the network 22, performs a process in response to a request from the endoscope part management device main body 68, and provides a processing result to the endoscope part management device main body 68.

The endoscope part management device main body 68 anonymizes the endoscopy information 44 and transmits the anonymized endoscopy information 44 and washing information 66 to the support management server 74. The anonymization of the endoscopy information 44 means the anonymization of the subject specified based on the subject ID included in the endoscopy information 44.

The support management server 74 receives the endoscopy information 44 and the washing information 66, and stores the received endoscopy information 44 and the washing information 66 in the management information storage device 76. The support management server 74 selectively acquires the endoscopy information 44 and the washing information 66 from the management information storage device 76 as necessary, and executes a process by using the acquired information.

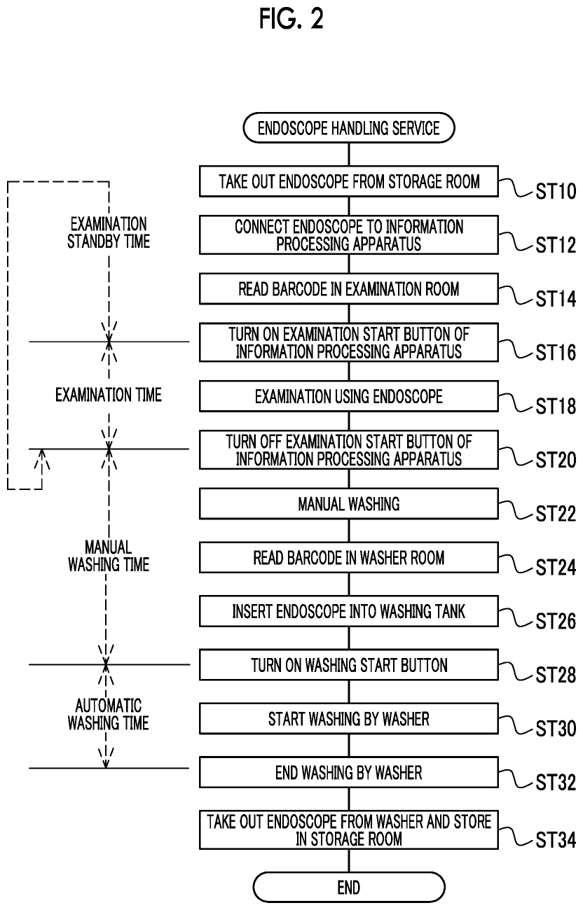

FIG. 2 shows an example of a flow of an endoscope handling service performed by the endoscopy worker. In the following, for convenience of explanation, the description is based on the premise that the usable endoscope 24 (for example, the endoscope 24 washed by the washer 52) is stored in a storage room (not shown) in a medical installation facility by the endoscopy worker.

In the endoscope handling service shown in FIG. 2, first, in step ST10, the endoscope 24 is taken out from the storage room by the endoscopy worker, and the endoscope 24 is transported into one examination room 30 among the plurality of examination rooms 30.

In the next step ST12, the endoscope 24 is connected to the information processing apparatus 36 in the examination room 30 via the light source device 31 and the endoscope processor device 32.

In the next step ST14, the barcode 42 of the endoscope 24 is read by the barcode reader 40 in the examination room 30. Accordingly, the endoscope ID of the endoscope 24 is acquired by the information processing apparatus 36.

In the next step ST16, the examination start button 38A of the information processing apparatus 36 is turned on. Further, an examination start button (not shown) is also provided in the endoscope processor device 32, and the examination start button of the endoscope processor device 32 is turned on. Accordingly, an endoscopy is started. Here, although an example of the embodiment has been described in which a process of step ST14 is performed prior to a process of step ST16, this is merely an example, and the process of step ST16 may be performed prior to the process of step ST14. That is, the order of step ST14 and step ST16 may be reversed.

In the following, for convenience of explanation, although the description is based on the premise that an endoscopy is started on a condition that the examination start button 38A is turned on, the present disclosed technology is not limited to this. For example, the endoscope processor device 32 may also include an examination start button similar to the examination start button 38A of the information processing apparatus 36, and the endoscopy may be started on a condition that the examination start button 38A of the information processing apparatus 36 is turned on and the examination start button of the endoscope processor device 32 is also turned on. In this case, the endoscope processor device 32 and the information processing apparatus 36 are examples of the “information processing apparatuses” according to the present disclosed technology.

In the next step ST18, an examination using the endoscope 24 (such as imaging of a test observation site by the imaging part 24B of the endoscope 24) is performed by a doctor or the like. Accordingly, the information processing apparatus 36 generates an image based on an imaging signal obtained by imaging by the imaging part 24B.

In the next step ST20, the examination start button 38A of the information processing apparatus 36 is turned off. Further, the examination start button of the endoscope processor device 32 is also turned off. Accordingly, the endoscopy is ended. The time during which the information processing apparatus 36 is being operated corresponds to time from when the examination start button 38A is turned on in step ST16 to when the examination start button 38A is turned off in step ST20, and is time (hereinafter, also referred to as “examination time”) during which the endoscopy is being performed.

In the following, for convenience of explanation, although the description is based on the premise that the endoscopy is ended on a condition that the examination start button 38A is turned off, the present disclosed technology is not limited to this. For example, the endoscopy may end on a condition that the examination start button 38A of the information processing apparatus 36 is turned off and the examination start button of the endoscope processor device 32 is also turned off.

In the next step ST22, manual washing of the endoscope 24 used in step ST18 is performed. In the next step ST24, the barcode 42 of the endoscope 24 manually washed in the washer room 50 is read by the barcode reader 64. Accordingly, the endoscope ID of the endoscope 24 is acquired by the washer management device 54.

In the next step ST26, the endoscope 24 is inserted into the washing tank of the washer 52. In the next step ST28, the washing start button 60A is turned on.

Note that time from when the endoscopy is ended to when washing of the endoscope 24 by the washer 52 is started, that is, time from when the examination start button 38A is turned off in step ST20 to when the washing start button 60A is turned on in step ST28, is washing wait time to when washing of the endoscope 24 by the washer 52 is started. The washing wait time is also time during which manual washing is performed with respect to the endoscope 24. In the following, for convenience of explanation, the washing wait time, that is, the time during which manual washing is performed on the endoscope 24, is also referred to as “manual washing time”.

In the next step ST30, washing of the endoscope 24, which is inserted into the washing tank in step ST26, is started by the washer 52. In the next step ST32, after a lapse of designated time after the washing start button 60A is turned on in step ST28, the washing of the endoscope 24, which is inserted into the washing tank in step ST26, by the washer 52 is ended, and one endoscope handling service is ended.

The time from when the washing start button 60A is turned on in step ST28 to when the washing of the endoscope 24 by the washer 52 is ended in step ST32 is time corresponding to time during which washing of the endoscope 24 is being performed by the washer 52 (hereinafter, also referred to as “automatic washing time”).

In the next step ST34, the endoscope 24 is taken out from the washer 52, and the taken-out endoscope 24 is stored in the storage room.

In a case where N is a natural number, in a specific examination room 30, time required from immediately before a time when an N-th endoscopy is ended (immediately before a time when the examination start button 38A is turned off in step ST20 included in the N-th endoscope handling service) to immediately before a time when an (N+1)-th endoscopy is started (immediately before a time when the examination start button 38A is turned on in step ST16 included in the (N+1)-th endoscope handling service) is time corresponding to time during which the endoscopy is on standby in a specific examination room 30 (hereinafter, also referred to as “examination standby time”). Here, the N-th endoscopy is an example of a “first endoscope procedure service that is the N-th endoscope procedure service” according to the present disclosed technology, and the (N+1)-th endoscopy is an example of a “second endoscope procedure service that is the (N+1)-th endoscope procedure service” according to the present disclosed technology.

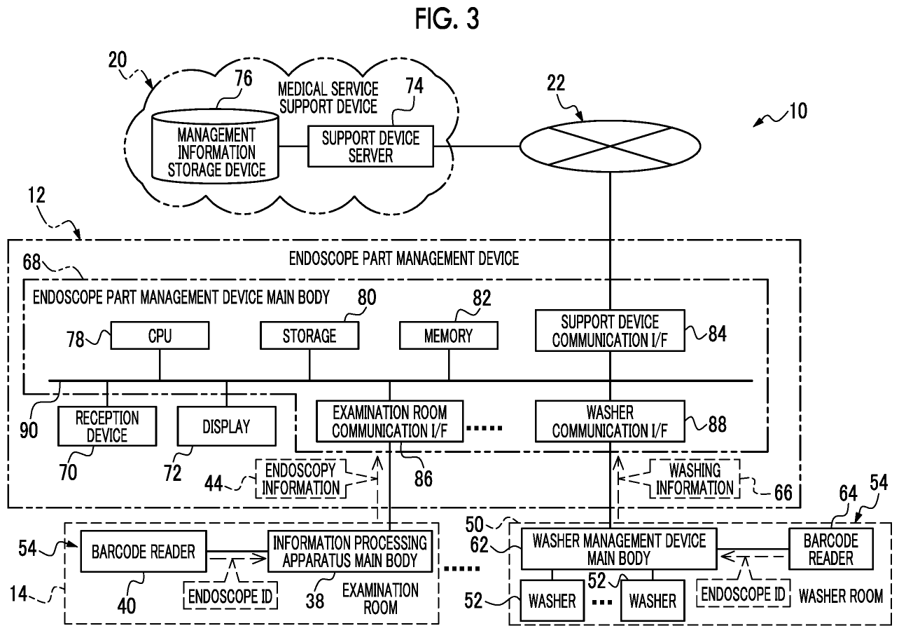

As an example, as shown in FIG. 3, the endoscope part management device main body 68 includes a CPU 78, a storage 80, a memory 82, a support device communication (interface) I/F 84, a plurality of examination room communication I/Fs 86, and a washer communication I/F 88. The CPU 78, the storage 80, the memory 82, the support device communication I/F 84, the plurality of examination room communication I/Fs 86, and the washer communication I/F 88 are connected to a bus 90.

The CPU 78 controls the entire endoscope part management device main body 68. The storage 80 is a non-volatile storage device that stores various programs, various parameters, and the like. Examples of the storage 80 include an EEPROM, an SSD, and/or an HDD. The memory 82 is a memory in which information is temporarily stored and is used as a work memory by the CPU 78. Examples of the memory 82 include a RAM.

The support device communication I/F 84 is connected to the network 22 and controls the exchange of information between the CPU 78 and the support management server 74. For example, the support device communication I/F 84 transmits information in response to a request from the CPU 78 to the support management server 74 via the network 22, receives the information transmitted from the support management server 74, and outputs the received information to the CPU 78.

The examination room communication I/F 86 is provided for each examination room 30 and is connected to the information processing apparatus main body 38 in the examination room 30. The examination room communication I/F 86 controls the exchange of information between the CPU 78 and the information processing apparatus main body 38. For example, the examination room communication I/F 86 transmits information in response to a request from the CPU 78 to the information processing apparatus main body 38 designated by the CPU 78 among the plurality of information processing apparatus main bodies 38. Further, the examination room communication I/F 86 acquires the endoscopy information 44 from the information processing apparatus main body 38 and outputs the acquired endoscopy information 44 to the CPU 78.

The washer communication I/F 88 is connected to a washer management device main body 62 and controls the exchange of information between the CPU 78 and the washer management device main body 62. For example, the washer communication I/F 88 transmits information in response to a request from the CPU 78 to the washer management device main body 62. Further, the washer communication I/F 88 acquires the washing information 66 from the washer management device main body 62 and outputs the acquired washing information 66 to the CPU 78.

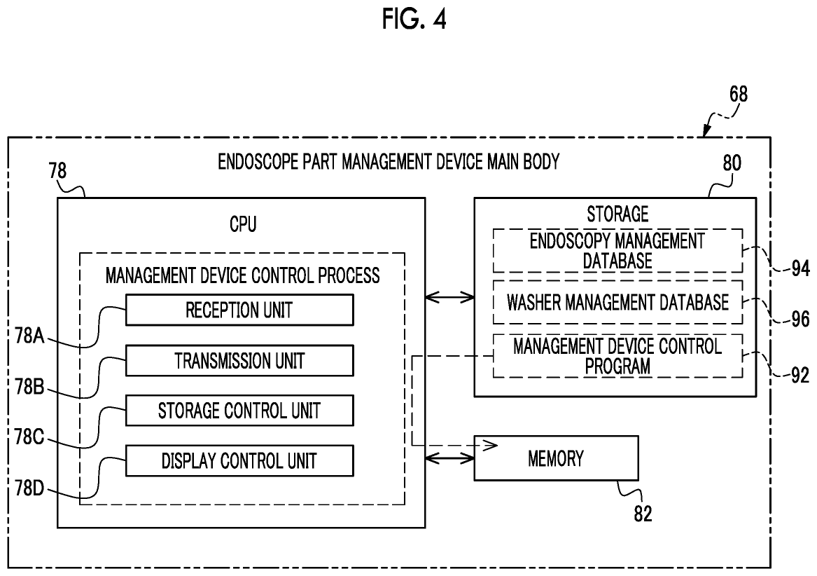

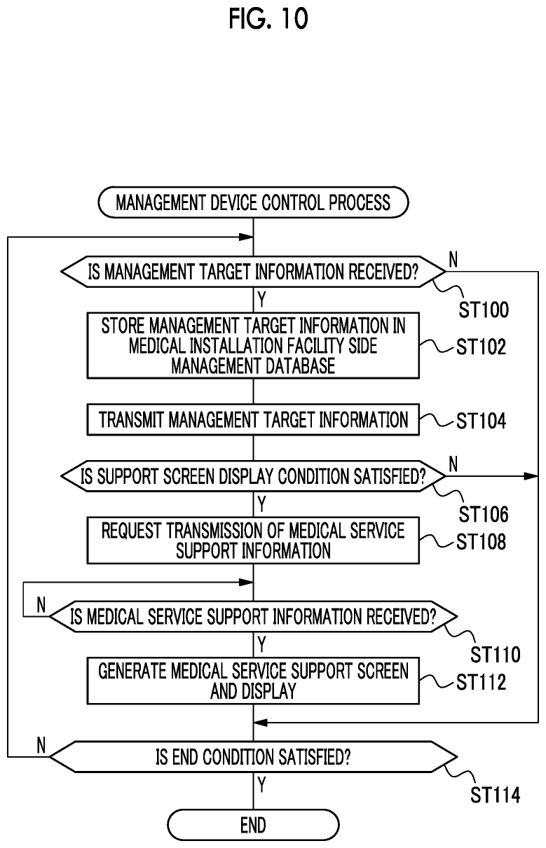

As an example, as shown in FIG. 4, in the endoscope part management device main body 68, the storage 80 stores a management device control program 92. The CPU 78 reads the management device control program 92 from the storage 80 and performs a management device control process by executing the read management device control program 92 on the memory 82 (see FIG. 10). The management device control process is realized by the CPU 78 operating as a reception unit 78A, a transmission unit 78B, a storage control unit 78C, and a display control unit 78D in accordance with the management device control program 92. Specific processing contents of the reception unit 78A, the transmission unit 78B, the storage control unit 78C, and the display control unit 78D will be described later with reference to FIG. 10.

An endoscopy management database 94 and a washer management database 96 are constructed in the storage 80. The storage control unit 78C acquires the endoscopy information 44 (see FIG. 1 and FIG. 3) via the examination room communication I/F 86 (see FIG. 3) and stores the acquired endoscopy information 44 in the endoscopy management database 94. Further, the storage control unit 78C acquires the washing information 66 (see FIG. 1 and FIG. 3) via the washer communication I/F 88 (see FIG. 3) and stores the acquired washing information 66 in the washer management database 96. In the following, for convenience of explanation, in a case where it is not necessary to distinguish between the endoscopy management database 94 and the washer management database 96, a term “medical installation facility side management database” will also be used.

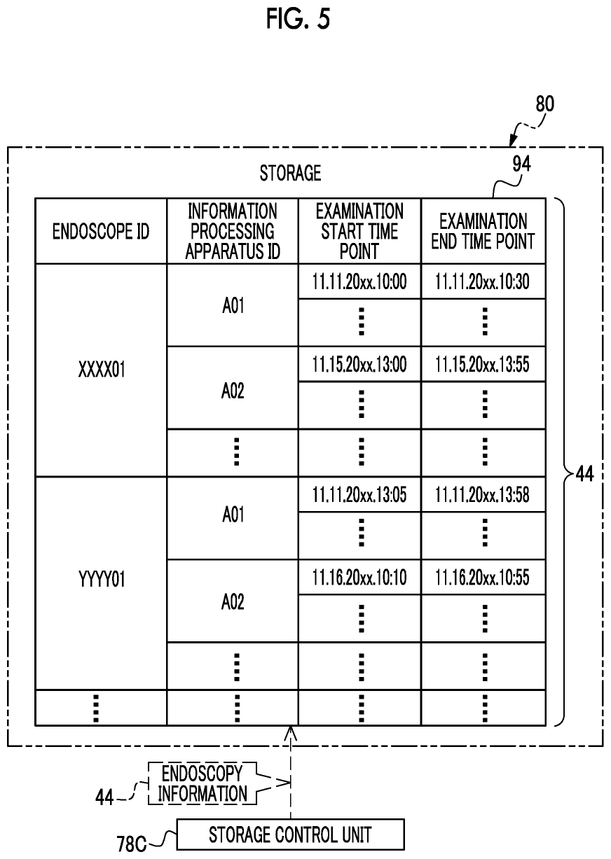

As an example, as shown in FIG. 5, in the endoscopy management database 94, the endoscopy information 44 acquired by the storage control unit 78C from each information processing apparatus main body 38 installed in each of the plurality of examination rooms 30 is stored. In the endoscopy management database 94, the examination start time point and the examination end time point are associated for each endoscope ID and for each information processing apparatus ID. Further, the subject ID is associated with the examination start time point or the examination end time point (not shown).

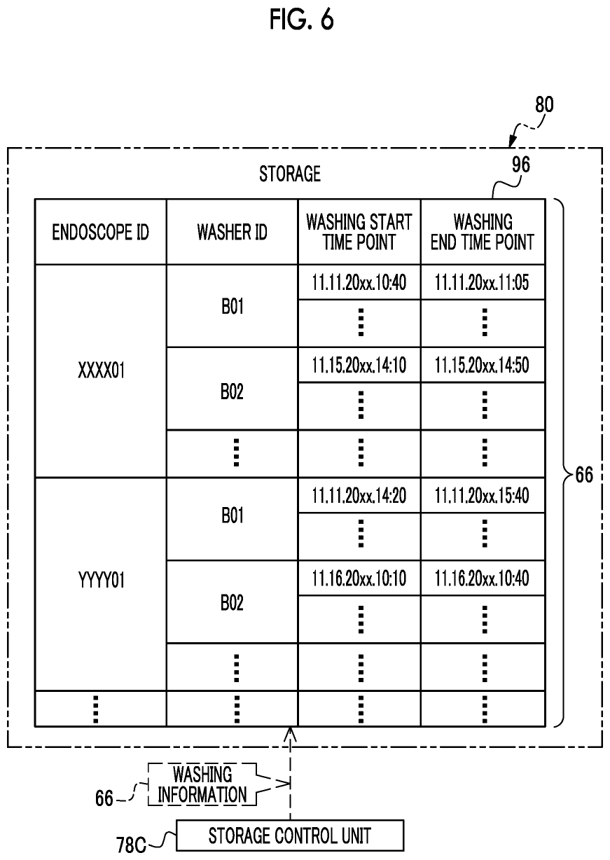

As an example, as shown in FIG. 6, in the washer management database 96, the washing information 66 acquired by the storage control unit 78C from the washer management device main body 62 is stored. In the washer management database 96, the washing start time point and the washing end time point are associated for each endoscope ID and for each washer ID.

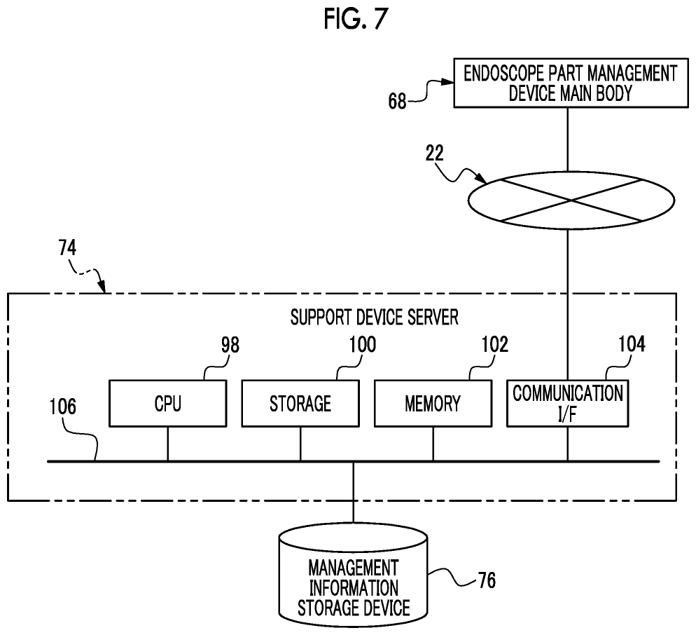

As an example, as shown in FIG. 7, the support management server 74 includes a CPU 98, a storage 100, a memory 102, and a communication I/F 104. The CPU 98 is an example of a “processor” according to the present disclosed technology, and the memory 102 is an example of a “memory” according to the present disclosed technology.

The CPU 98, the storage 100, the memory 102, and the communication I/F 104 are connected to a bus 106. Further, the management information storage device 76 is connected to the bus 106. Examples of the management information storage device 76 include an EEPROM, an SSD, and/or an HDD. In the management information storage device 76, anonymized management target information is stored.

The CPU 98 controls the entire support management server 74. The storage 100 is a non-volatile storage device that stores various programs, various parameters, and the like. Examples of the storage 100 include an EEPROM, an SSD, and/or an HDD. The memory 102 is a memory in which information is temporarily stored and is used as a work memory by the CPU 98. Examples of the memory 102 include a RAM.

The communication I/F 104 is connected to the endoscope part management device main body 68 via the network 22 and controls the exchange of information between the CPU 98 and the endoscope part management device main body 68. For example, the communication I/F 104 receives the information transmitted from the endoscope part management device main body 68 and outputs the received information to the CPU 78. Further, the communication I/F 104 transmits information in response to a request from the CPU 98 to the endoscope part management device main body 68 via the network 22.

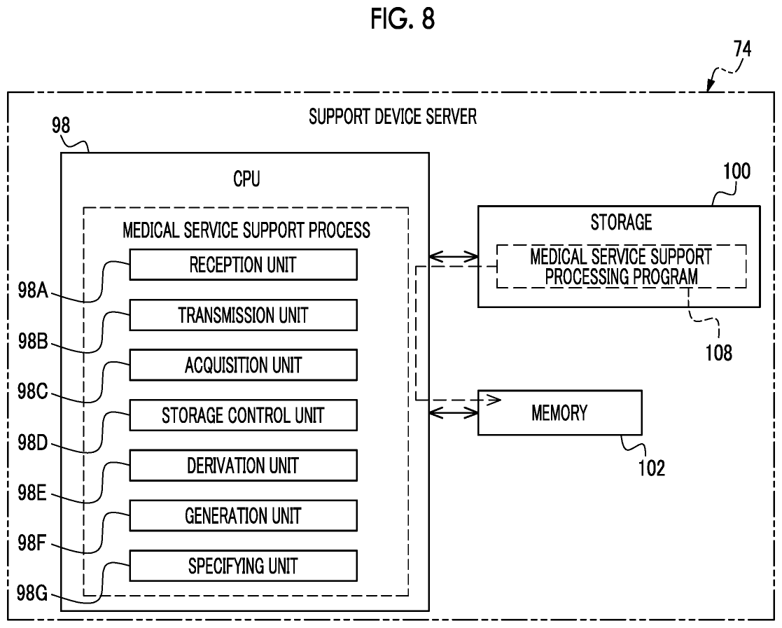

As an example, as shown in FIG. 8, in the support management server 74, the storage 100 stores a medical service support processing program 108. The support management server 74 is an example of a “computer” according to the present disclosed technology, and the medical service support processing program 108 is an example of a “program” according to the present disclosed technology.

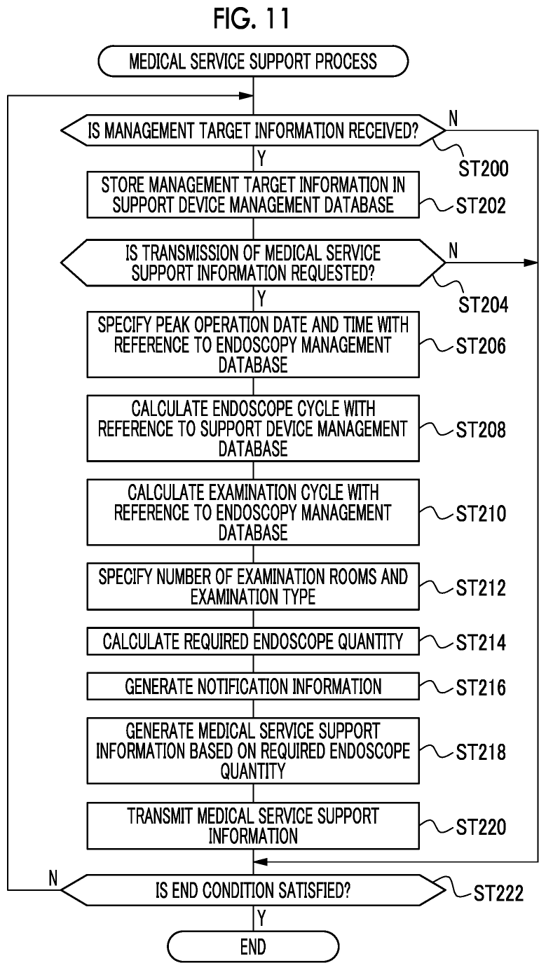

The CPU 98 reads the medical service support processing program 108 from the storage 100 and performs a medical service support process by executing the read medical service support processing program 108 on the memory 102 (see FIG. 11). The medical service support process is realized by the CPU 98 operating as a reception unit 98A, a transmission unit 98B, an acquisition unit 98C, a storage control unit 98D, a derivation unit 98E, a generation unit 98F, a specifying unit 98G in accordance with the medical service support processing program 108. Specific processing contents of the reception unit 98A, the transmission unit 98B, the acquisition unit 98C, the storage control unit 98D, the derivation unit 98E, the generation unit 98F, the specifying unit 98G will be described later with reference to FIG. 11.

By performing the medical service support process, the CPU 98 derives a required quantity of the endoscopes 24 (hereinafter, also simply referred to as the “required endoscope quantity”) based on an examination cycle and an endoscope cycle and outputs the medical service support information based on the derived required endoscope quantity.

Here, the examination cycle refers to the sum of the average value of the examination time required for the endoscopy and the shortest standby time selected as the shortest time of the examination standby time (see FIG. 2). Further, the endoscope cycle refers to the sum of the average value of the examination time required for the endoscopy and the average value of the washing time from when the endoscopy is ended to when washing of the endoscope 24 is ended. Here, the average values of the washing times from when the endoscopy is ended to when washing of the endoscope 24 is ended is the sum of the average value of the manual washing times (see FIG. 2) and the average value of the automatic washing times (see FIG. 2). For convenience of explanation, hereinafter, the average value of the examination time required for an endoscopy is also simply referred to as “average examination time”, the average value of the washing time from when the endoscopy is ended to when washing of the endoscope 24 is ended is also simply referred to as “average washing time”, the average value of the manual washing time is also simply referred to as “average manual washing time”, and the average value of the automatic washing time is also simply referred to as “average automatic washing time”.

Note that the average examination time is an example of the “standard procedure service time” according to the present disclosed technology, the shortest standby time is an example of the “standard standby time” according to the present disclosed technology, and the average washing time is an example of the “standard washing time” according to the present disclosed technology. Further, the average manual washing time is an example of “first washing time” according to the present disclosed technology, and the average automatic washing time is an example of “second washing time” according to the present disclosed technology.

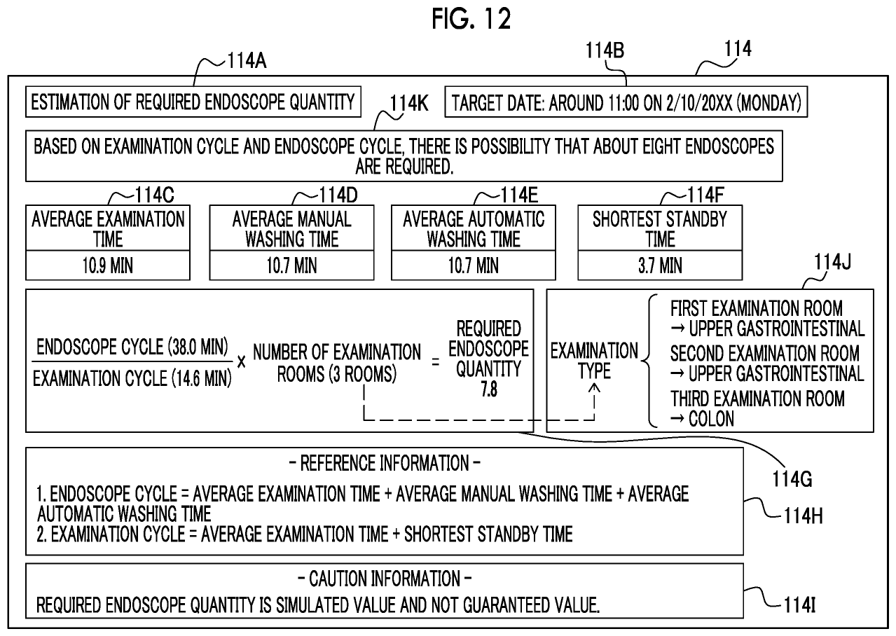

As described in detail later, the medical service support information is information that includes information capable of specifying a required endoscope quantity. In the medical service support information, screen information that indicates a medical service support screen 114 (see FIG. 12) is included as information including information that can specify the required endoscope quantity.

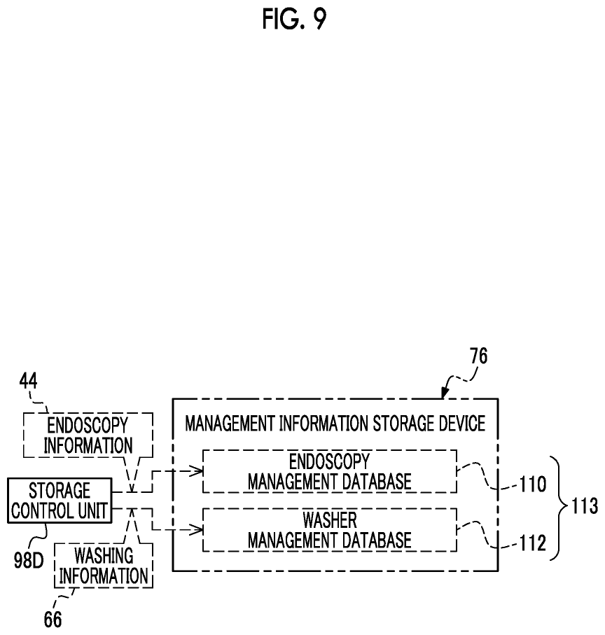

As an example, as shown in FIG. 9, a support device management database 113 is constructed in the management information storage device 76. The support device management database 113 includes an endoscopy management database 110 and a washer management database 112. The anonymized endoscopy information 44 is stored in the endoscopy management database 110, and the washing information 66 is stored in the washer management database 112.

The storage control unit 98D acquires the anonymized endoscopy information 44 from the endoscope part management device main body 68 via the communication I/F 104 (see FIG. 7) and stores the acquired endoscopy information 44 in the endoscopy management database 110. The storage control unit 98D acquires the washing information 66 (see FIG. 6) from the endoscope part management device main body 68 via the communication I/F 104 (see FIG. 7) and stores the acquired washing information 66 in the washer management database 112.

Next, the operation of the medical service support system will be described.

FIG. 10 shows an example of a flow of a management device control process performed by the CPU 78 of the endoscope part management device 12. In the present embodiment, the information processing apparatus 36 will be described below as an example of the medical device.

In the management device control process shown in FIG. 10, first, in step ST100, the reception unit 78A determines whether or not the management target information is received through the examination room communication I/F 86 (see FIG. 3) or the washer communication I/F 88 (see FIG. 3). In step ST100, in a case in which the management target information is not received through the examination room communication I/F 86 or the washer communication I/F 88, a negative determination is made, and the management device control process proceeds to step ST114. In step ST100, in a case in which the management target information is received through the examination room communication I/F 86 or the washer communication I/F 88, a positive determination is made, and the management device control process proceeds to step ST102.

In step ST102, the storage control unit 78C stores the management target information, which is received through the examination room communication I/F 86 or the washer communication I/F 88 in step ST100, in the medical installation facility side management database. That is, the endoscopy information 44 is stored in the endoscopy management database 94 (see FIGS. 4 and 5), and the washing information 66 is stored in the washer management database 96 (see FIGS. 4 and 6). After the process in step ST102 is executed, the management device control process proceeds to step ST104.

In step ST104, the transmission unit 78B transmits the management target information, which is received in step ST100, to the support management server 74 via the support device communication I/F 84 (see FIG. 3). After the process in step ST104 is executed, the management device control process proceeds to step ST106.

In step ST106, the transmission unit 78B determines whether or not a support screen display condition, which is a condition for displaying the medical service support screen 114 (see FIG. 12) on the display 72 (see FIG. 1 and FIG. 3), is satisfied. A first example of the support screen display condition includes a condition that the reception device 70 (see FIGS. 1 and 3) receives an instruction for displaying the medical service support screen 114 on the display 72. Further, a second example of the support screen display condition includes a condition that a time point (for example, 22:00), which is designated in advance, has arrived. Further, the second example of the support screen display condition includes a condition that an amount of information of the management target information stored in the medical installation facility side management database reaches a default amount of information.

In step ST106, in a case where the support screen display condition is not satisfied, a negative determination is made, and the management device control process proceeds to step ST114. In step ST106, in a case where the support screen display condition is satisfied, a positive determination is made, and the management device control process proceeds to step ST108.

In step ST108, the transmission unit 78B requests transmission of the medical service support information from the support management server 74 via the support device communication I/F 84 (see FIG. 3). After the process in step ST108 is executed, the management device control process proceeds to step ST110.

In a case where the process of step ST108 is executed, the processes of step ST206 to step ST222, which are included in the medical service support process shown in FIG. 11, are executed by the support management server 74, and the medical service support information is transmitted from the support management server 74 to the endoscope part management device 12 via the network 22 by executing step ST220.

In step ST110, the reception unit 78A determines whether or not the medical service support information, which is transmitted from the support management server 74, is received through the support device communication I/F 84 (see FIG. 3). In step ST110, in a case where the medical service support information is not received through the support device communication I/F 84, a negative determination is made, and the determination in step ST110 is performed again. In step ST110, in a case in which the medical service support information is received through the support device communication I/F 84, a positive determination is made, and the management device control process proceeds to step ST112.

In step ST112, the display control unit 78D generates the medical service support screen 114 (see FIG. 12) by using the medical service support information received through the support device communication I/F 84 in step ST110 and displays the medical service support screen 114 (see FIG. 12) on the display 72 (see FIG. 1 and FIG. 3). After the process in step ST112 is executed, the management device control process proceeds to step ST114.

In step ST114, the display control unit 78D determines whether or not a condition for ending the management device control process (hereinafter, referred to as a “management device control process end condition”) is satisfied. Examples of the management device control process end condition include a condition that an instruction for ending the management device control process is received by the reception device 70 (see FIG. 1 and FIG. 3).

In step ST114, in a case where the management device control process end condition is not satisfied, a negative determination is made, and the management device control process proceeds to step ST100. In step ST114, in a case where the management device control process end condition is satisfied, the management device control process is ended.

FIG. 11 shows an example of a flow of the medical service support process performed by the CPU 98 of the support management server 74.

In the medical service support process shown in FIG. 11, first, in step ST200, the reception unit 98A determines whether or not the management target information, which is transmitted by executing the process of step ST100 included in the management device control process shown in FIG. 10, is received through the communication I/F 104 (see FIG. 7). In step ST200, in a case where the management target information is not received through the communication I/F 104, a negative determination is made, and the medical service support process proceeds to step ST222. In step ST200, in a case where the management target information is received through the communication I/F 104, a positive determination is made, and the medical service support process proceeds to step ST202.

In step ST202, the storage control unit 98D stores the management target information, which is received through the communication I/F 104 in step ST200, in the support device management database 113. That is, the endoscopy information 44 is stored in the endoscopy management database 110 (see FIG. 9), and the washing information 66 is stored in the washer management database 112 (see FIG. 9). After the process in step ST202 is executed, the medical service support process proceeds to step ST204.

In step ST204, the reception unit 98A determines whether or not transmission of the medical service support information is requested from the endoscope part management device 12 by executing the process of step ST108 included in the management device control process shown in FIG. 10. In step ST204, in a case where the transmission of the medical service support information is not requested from the endoscope part management device 12, a negative determination is made, and the medical service support process proceeds to step ST222. In step ST204, in a case where the transmission of the medical service support information is requested from the endoscope part management device 12, a positive determination is made, and the medical service support process proceeds to step ST206.

In step ST206, the specifying unit 98G specifies a date on which an operation status of the medical device peaks (hereinafter, also referred to as “peak operation date”) and specifies an examination time slot in which the operation status of the medical device peaks on the peak operation date (hereinafter, also referred to as “peak operation time slot”), with reference to the endoscopy management database 110 (see FIG. 9).

In the present embodiment, the peak operation date refers to a date on which the operation statuses of all the information processing apparatuses 36 (hereinafter, also referred to as “all information processing apparatuses 36”) installed in all the examination rooms 30 peak within a period designated in advance (for example, a period designated by the user or the like). Further, in the present embodiment, the peak operation time slot refers to an examination time slot in which the operation statuses of all the information processing apparatuses 36 peak on the peak operation date. Hereinafter, for convenience of explanation, the peak operation date and the peak operation time slot are also referred to as “peak operation date and time”. The peak operation date and time is an example of “a time period when the operation degree of the medical device, which is used in the endoscope handling service including the endoscope procedure service, is equal to or higher than a reference degree” according to the present disclosed technology. The operation degree of the medical device indicates information that indicates a ratio of operation of the medical device that can be acquired by the medical service support device and is indicated by, for example, a value based on the number of endoscopies. The value based on the number of endoscopies indicates, for example, the number of endoscopies for each predetermined period such as for each day or for each hour. Further, the value based on the number of endoscopies indicates a total number of endoscopies, which are executed by a plurality of medical devices, for each predetermined period in a case where there are a plurality of medical devices that can be acquired by the medical service support device. The value based on the number of endoscopies may be obtained from the information of the examination start time point and the examination end time point of each endoscopy with reference to the endoscopy management database. The reference degree is a reference value. More specifically, it is the reference value for determining a period during which endoscopy is intensively performed, such as the peak operation date or the peak operation time. The reference value may be a fixed value set in advance, or a variable value changed according to a user input. Further, the reference value may be a variation value calculated based on the number of endoscopies performed in the past. The reference value may be, for example, an average value of the number of endoscopies performed in the past or may be the maximum value of the number of endoscopies performed in the past or a value obtained by adding or multiplying the maximum value by a predetermined value. Further, in a case where the operation degree of the medical device indicates the number of endoscopies for each first period (for example, one day), the highest value, which is within the number of endoscopies for each first period in a second period (for example, one week) longer than the first period, may be defined as the reference value.

Here, a date on which an endoscopy is performed the highest number of times is regarded as the peak operation date. Further, the examination time slot in which an average value of the operation time of all the information processing apparatuses 36 on the peak operation date is the maximum is defined as the peak operation time slot. The peak operation date and time obtained in this manner is an example of “a time period when a value based on the number of endoscope procedure services is equal to or greater than the reference value” according to the present disclosed technology. Further, although the peak operation date and the peak operation date and time are exemplified here, this is merely an example, and a date or a date and time designated by the user or the like may be used instead of the peak operation date and the peak operation date and time.

The date on which an endoscopy is performed can be specified by using the examination start time point or the examination end time point of the endoscopy information 44 stored in the endoscopy management database 110. Therefore, in the present step ST206, the date on which an endoscopy is performed the highest number of times is specified by the specifying unit 98G by using the examination start time point or the examination end time point of the endoscopy information 44 included in the endoscopy management database 110. Further, the operation time of all the information processing apparatuses 36 can be specified by using the examination start time point and the examination end time point of the endoscopy information 44 stored in the endoscopy management database 110. Therefore, in the present step ST206, the peak operation time slot is specified by the specifying unit 98G by using the examination start time point and the examination end time point of the endoscopy information 44 included in the endoscopy management database 110.

The peak operation date may be specified for all periods that can be specified based on the endoscopy information 44 stored in the endoscopy management database 110 or may be a period (for example, a designated number of years, a designated number of months, or a designated number of days) designated based on the endoscopy information 44 stored in the endoscopy management database 110. The designation of the period is performed, for example, by a user or the like via the reception device 70 (see FIG. 1 and FIG. 3) of the endoscope part management device 12.

Further, the peak operation time slot is not limited to the average value of the operation time of all the information processing apparatuses 36, and the peak operation time slot may be an examination time slot in which the quantity of the information processing apparatuses 36 which are in operation is the highest, may be an examination time slot in which the median value of the operation time of the information processing apparatuses 36 is the maximum, and may be an examination time slot selected from a value based on the quantity of information processing apparatuses 36 and/or a statistical value of the operation time.

Further, although the peak operation date and time is exemplified here, this is merely an example, and the peak operation date may be applied instead of the peak operation date and time.

Next, in step ST208, the derivation unit 98E calculates the endoscope cycle for the peak operation date and time specified in step ST206, with reference to the support device management database 113 (see FIG. 9). After the process in step ST208 is executed, the medical service support process proceeds to step ST210.

The endoscope cycle, which is calculated in the present step ST208, is the sum of the average examination time of the peak operation date and time specified in step ST206, the average manual washing time of the peak operation date and time specified in step ST206, and the average automatic washing time of the peak operation date and time specified in step ST206.

In the present step ST208, the average examination time included in the endoscope cycle is an average value of all the examination times corresponding to all the endoscopes 24 used in the peak operation date and time specified in step ST206. The examination time corresponding to the endoscope 24 used on the peak operation date and time specified in step ST206 is calculated by the derivation unit 98E based on the examination start time point and the examination end time point, which are associated with the endoscope ID related to the endoscope 24 used on the peak operation date and time specified in step ST206, among all the examination start time points and all the examination end time points included in the endoscopy information 44 (see FIG. 5) stored in the endoscopy management database 110.

In the present step ST208, the average manual washing time included in the endoscope cycle is an average value of all the manual washing times corresponding to all the endoscopes 24 used in the peak operation date and time specified in step ST206. The manual washing time corresponding to the endoscope 24 used on the peak operation date and time specified in step ST206 is calculated by the derivation unit 98E based on the examination end time point and the washing start time point. The examination end time point, which is used to calculate the manual washing time, is the examination end time point associated with the endoscope ID related to the endoscope 24 used on the peak operation date and time specified in step ST206, among all the examination end time points included in the endoscopy information 44 (see FIG. 5) stored in the endoscopy management database 110. Further, the washing start time point, which is used to calculate the manual washing time, is the washing start time point associated with the endoscope ID related to the endoscope 24 used on the peak operation date and time specified in step ST206, among all the washing start time points included in the washing information 66 (see FIG. 6) stored in the washer management database 112.

In the present step ST208, the average automatic washing time included in the endoscope cycle is an average value of all the automatic washing times corresponding to all the endoscopes 24 used in the peak operation date and time specified in step ST206. The automatic washing time corresponding to the endoscope 24 used on the peak operation date and time specified in step ST206 is calculated by the derivation unit 98E based on the washing start time point and the washing end time point, which are associated with the endoscope ID related to the endoscope 24 used on the peak operation date and time specified in step ST206, among all the washing start time points and washing end time points included in the washing information 66 (see FIG. 6) stored in the washer management database 112.

Here, although the sum of the average examination time, the average manual washing time, and the average automatic washing time is exemplified as an example of the endoscope cycle, the present disclosed technology is not limited to this, and the endoscope cycle may be the sum of the average examination time, the average manual washing time, the average automatic washing time, and the shortest standby time.

In step ST210, the derivation unit 98E calculates the examination cycle for the peak operation date and time specified in step ST206, with reference to the endoscopy management database 110 (see FIG. 9). After the process in step ST210 is executed, the medical service support process proceeds to step ST212.

The examination cycle, which is calculated in the present step ST210, is the sum of the average examination time of the peak operation date and time specified in step ST206 and the shortest standby time of the peak operation date and time specified in step ST206.

In the present step ST210, the average examination time included in the examination cycle is the same as the average examination time included in the endoscope cycle of step ST208.

In the present step ST210, the shortest standby time included in the examination cycle is calculated by the derivation unit 98E based on all the examination start time points and examination end time points corresponding to all the endoscope IDs included in the endoscopy information 44 (see FIG. 5) stored in the endoscopy management database 110. That is, for all the endoscopes 24, the shortest time is used as the shortest standby time included in the examination cycle, out of times from the examination end time point related to the N-th endoscopy to the examination start time point related to the (N+1)-th endoscopy.

In step ST212, the specifying unit 98G specifies the number of rooms of the examination room 30 (hereinafter, also referred to as “the number of examination rooms”) in which endoscopy is being performed on the peak operation date and time specified in step ST206 and specifies the type of endoscopy (hereinafter, also referred to as “examination type”) being performed on the peak operation date and time specified in step ST206. After the process in step ST212 is executed, the medical service support process proceeds to step ST214.

In the present step ST212, the number of examination rooms coincides with the quantity of information processing apparatuses 36 being operated on the peak operation date and time specified in step ST206. This is because one information processing apparatus 36 is installed in each of the examination rooms 30. Therefore, the specifying unit 98G specifies the number of examination rooms by specifying the number of information processing apparatus IDs corresponding to the peak operation date and time specified in step ST206, among all the information processing apparatus IDs based on the endoscopy information 44 stored in the endoscopy management database 110. That is, the number of information processing apparatus IDs is recognized by the specifying unit 98G as the number of examination rooms.

In the present step ST212, the examination type is derived, for example, from an examination type derivation table (not shown) by the specifying unit 98G. For example, the examination type derivation table is a table in which the endoscope ID and the endoscopy type information are associated with each other. The endoscopy type information refers to information indicating a type of endoscopy in which the endoscope 24 that is specified based on the endoscope ID is used. The type of endoscopy means, for example, an application of the endoscope 24. Examples of the type of endoscopy include upper gastrointestinal examination, pancreatic duct examination, bile duct examination, duodenal examination, colon examination, intracerebral examination, otolaryngology examination, bronchial examination, oral endoscopy, nasal endoscopy, and the like.

The specifying unit 98G specifies the type of endoscopy performed on the peak operation date and time specified in step ST206 by deriving the endoscopy type information, which corresponds to the endoscope ID associated with the peak operation date and time specified in step ST206, from the examination type derivation table.

Here, although an example of the embodiment has been described in which the specifying unit 98G specifies the type of endoscopy by using the examination type derivation table, this is merely an example, and the specifying unit 98G may specify the type of endoscopy based on the endoscopy information 44. In this case, for example, the endoscopy type information may be associated with the examination start time point and/or the examination end time point of the endoscopy information 44 by the endoscope part management device 12 or the information processing apparatus 36. Accordingly, the specifying unit 98G can specify the type of endoscopy based on the endoscopy type information associated with the examination start time point and/or examination end time point corresponding to the peak operation date and time specified in step ST206.

In step ST214, the derivation unit 98E calculates the required endoscope quantity based on the endoscope cycle calculated in step ST208, the examination cycle calculated in step ST210, and the number of examination rooms specified in step ST212. After the process in step ST214 is executed, the medical service support process proceeds to step ST216.

In the present step ST214, the required endoscope quantity is derived by the derivation unit 98E based on a ratio of the endoscope cycle to the examination cycle. Specifically, the required endoscope quantity is calculated by using the expression “{(endoscope cycle)/(examination cycle)} x number of examination rooms”. That is, the required endoscope quantity is a value obtained by multiplying a ratio of the endoscope cycle calculated in step ST208 to the examination cycle calculated in step ST210 by the number of examination rooms specified in step ST212. Further, in the present step ST214, the number of examination rooms is an example of “the number of medical sites where an endoscope procedure service is performed” according to the present disclosed technology.

Further, in the present step ST214, although the ratio of the endoscope cycle calculated in step ST208 to the examination cycle calculated in step ST210 is exemplified, in at least one of the examination cycle or the endoscope cycle used herein, time for fine adjustment may be added or may be reduced, or a coefficient for fine adjustment may be multiplied.

In step ST216, the generation unit 98F generates notification information in which the required endoscope quantity, which is calculated in step ST214, is notified of. The notification information is visualized and displayed in the medical service support screen 114 (see FIG. 12). After the process in step ST216 is executed, the medical service support process proceeds to step ST218.

In step ST218, the generation unit 98F generates the medical service support information based on the required endoscope quantity calculated in step ST214. After the process in step ST218 is executed, the medical service support process proceeds to step ST220.

In the present step ST218, the medical service support information includes the peak operation date and time, the number of examination rooms, the examination type, the endoscope cycle, the examination cycle, the average examination time, the average manual washing time, the average automatic washing time, the shortest standby time, and the notification information.

The medical service support information is information that is capable of being represented via a comment, a figure, a table, and the like by a presentation device (here, the display 72 as an example) and includes screen information indicating the medical service support screen 114 (see FIG. 12). Here, although the screen information is generated by the medical service support device 20 as an example of the embodiment, this is merely an example, and the screen information may be generated by another device such as the endoscope part management device 12 or the like, for example.

In step ST220, the transmission unit 98B transmits the medical service support information, which is generated in step ST218, to the endoscope part management device main body 68 via the communication I/F 104 (see FIG. 7). After the process in step ST220 is executed, the medical service support process proceeds to step ST222.

In step ST222, the transmission unit 98B determines whether or not a condition for ending the medical service support process (hereinafter, referred to as a “medical service support process end condition”) is satisfied. Examples of the medical service support process end condition include a condition that an instruction for ending the medical service support process is received by the reception device (not shown).

In step ST222, in a case where the medical service support process end condition is not satisfied, a negative determination is made, and the medical service support process proceeds to step ST200. In step ST222, in a case where the medical service support process end condition is satisfied, a positive determination is made, and the medical service support process is ended.

In a case where the medical service support information is transmitted by executing the process of step ST220, as described above, the medical service support information is received through the support device communication I/F 84 of the endoscope part management device 12 (see step ST110 shown in FIG. 10). As shown in step ST112 shown in FIG. 10, the display control unit 78D of the endoscope part management device 12 generates the medical service support screen 114 (see FIG. 12) based on the medical service support information, and the generated medical service support screen 114 is displayed on the display 72 (see FIG. 12).

As an example, as shown in FIG. 12, on the medical service support screen 114, a screen title display field 114A, a required endoscope quantity calculation target date display field 114B, an average examination time display field 114C, an average manual washing time display field 114D, an average automatic washing time display field 114E, a shortest standby time display field 114F, a required endoscope quantity display field 114G, a reference information display field 114H, a caution information display field 114I, an examination type display field 114J, and an examination support comment display field 114K are displayed.

In the screen title display field 114A, a title that can enable recognition that the medical service support screen 114 is a screen displaying an estimate of the required endoscope quantity is displayed. In the example shown in FIG. 12, “estimation of required endoscope quantity” is displayed in the screen title display field 114A.

In the required endoscope quantity calculation target date display field 114B, the peak operation date and time, which is specified in step ST206 shown in FIG. 11, is displayed. In the example shown in FIG. 12, the peak operation date and time is represented by the year, month, date, day of the week, and time slot, such as “target date: around 11:00 on 2/10/20XX (Monday)”.

In the average examination time display field 114C, the average examination time, which is used in the calculation of the endoscope cycle in step ST208 shown in FIG. 11 and in the calculation of the examination cycle in step ST210 shown in FIG. 11, is displayed. In the average manual washing time display field 114D, the average manual washing time, which is used in the calculation of the endoscope cycle in step ST208 shown in FIG. 11, is displayed. In the average automatic washing time display field 114E, the average automatic washing time, which is used in the calculation of the endoscope cycle in step ST208 shown in FIG. 11, is displayed. In the shortest standby time display field 114F, the shortest standby time, which is used in the calculation of the examination cycle in step ST210 shown in FIG. 11, is displayed.

In the required endoscope quantity display field 114G, the required endoscope quantity, which is calculated in step ST214 shown in FIG. 11, is displayed. Further, in the required endoscope quantity display field 114G, the expression used in the calculation of the required endoscope quantity, the endoscope cycle, the examination cycle, and the number of examination rooms are displayed.

In the reference information display field 114H, the reference information is displayed. In the example shown in FIG. 12, as an example of the reference information, an endoscope cycle calculation method (in the example shown in FIG. 12, “endoscope cycle=average examination time+average manual washing time+average automatic washing time”) and an examination cycle calculation method (in the example shown in FIG. 12, “examination cycle=average examination time+shortest standby time”) are shown.

In the caution information display field 114I, the caution information is displayed. In the example shown in FIG. 12, as an example of the caution information, a comment indicating that the required endoscope quantity, which is displayed in the required endoscope quantity display field 114G, is a simulated value and not a guaranteed value is displayed.

In the examination type display field 114I, information indicating the examination type, which is specified in step ST212 shown in FIG. 11, that is, the type of endoscopy which is performed in each examination room 30 on the peak operation date and time, is displayed for each examination room 30.

In the examination support comment display field 114K, a comment related to the required endoscope quantity calculated based on the examination cycle and the endoscope cycle is displayed. In the example shown in FIG. 12, in the examination support comment display field 114K, a comment such as “Based on the examination cycle and the endoscope cycle, there is a possibility that about eight endoscopes are required” is displayed.

As described above, in the medical service support device 20 according to the present embodiment, the required endoscope quantity is derived based on the average examination time, the average manual washing time, the average automatic washing time, and the shortest standby time (see step ST214 shown in FIG. 11). In the medical service support device 20, the medical service support information based on the derived required endoscope quantity is transmitted to the endoscope part management device 12 (see step ST220 shown in FIG. 11). In the endoscope part management device 12, the medical service support screen 114 is generated based on the medical service support information and is displayed on the display 72 (see step ST112 shown in FIG. 10). Since the medical service support screen 114 is presented to the user or the like in a state in which the medical service support information is visualized, the user or the like can ascertain the required endoscope quantity. Therefore, according to the present configuration, it is possible to support the efficient performance of the endoscope procedure service.

Further, the required endoscope quantity is calculated based on the number of examination rooms, the average examination time, the average manual washing time, the average automatic washing time, and the shortest standby time. Therefore, according to the present configuration, the user or the like can ascertain the highly accurate quantity as the required quantity of the endoscopes 24, as compared with a case where the required quantity of the endoscopes 24 is predicted by the user or the like only based on the average examination time, the average manual washing time, and the average automatic washing time.

Further, the required endoscope quantity is calculated based on a ratio of the endoscope cycle to the examination cycle, that is a ratio of the sum of the average examination time, the average manual washing time, and the average automatic washing time to the sum of the average examination time and the shortest standby time. Therefore, according to the present configuration, the user or the like can ascertain the highly accurate quantity as the required quantity of the endoscopes 24, as compared with a case where the required quantity of the endoscopes 24 is predicted by the user or the like only based on the examination cycle or the endoscope cycle.

Further, the endoscope cycle includes average manual washing time and average automatic washing time. Therefore, according to the present configuration, it is possible to obtain the required endoscope quantity with high accuracy as compared with a case where the endoscope cycle does not include at least one of the average manual washing time or the average automatic washing time.

Further, the medical service support information is visualized and presented as the medical service support screen 114 via the display 72. Therefore, according to the present configuration, it is possible for the user or the like to visually ascertain the medical service support information.

Further, the medical service support information includes the notification information in which the required endoscope quantity is notified of Since the medical service support information is visualized and presented as the medical service support screen 114 via the display 72, the required endoscope quantity is notified to the user or the like via the medical service support screen 114. Therefore, according to the present configuration, the user or the like can ascertain the required endoscope quantity.

Further, in the medical service support device 20, the required endoscope quantity on the peak operation date and time is derived. Further, in the endoscope part management device 12, the required endoscope quantity on the peak operation date and time is displayed on the medical service support screen 114. Therefore, according to the present configuration, the user or the like can ascertain the required endoscope quantity on the peak operation date and time.

Further, the peak operation date is the date on which an endoscopy is performed the highest number of times, and the peak operation time slot is the examination time slot in which the average value of the operation time of all the information processing apparatuses 36 on the peak operation date is the highest. Therefore, according to the present configuration, the user or the like can ascertain the required endoscope quantity for the examination time slot in which the average value of the operation time of all the information processing apparatuses 36 is the highest on the date on which an endoscopy is performed the highest number of times.

Further, in the embodiment described above, although an example of the embodiment has been described in which the medical service support information, which is based on the required endoscope quantity on the peak operation date and time, is generated and output by the CPU 98 of the support management server 74 regardless of the type of endoscopy, the present disclosed technology is not limited to this. For example, the CPU 98 of the support management server 74 may acquire the examination cycle and the endoscope cycle for each type of endoscopy, derive the required endoscope quantity for each type of endoscopy based on the examination cycle and the endoscope cycle, and output the medical service support information for each type of endoscopy based on the required endoscope quantity.

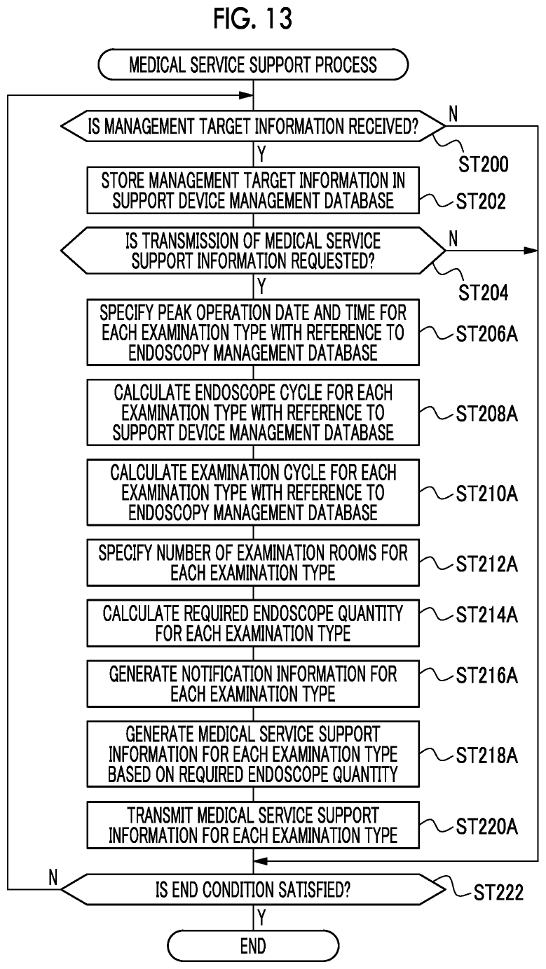

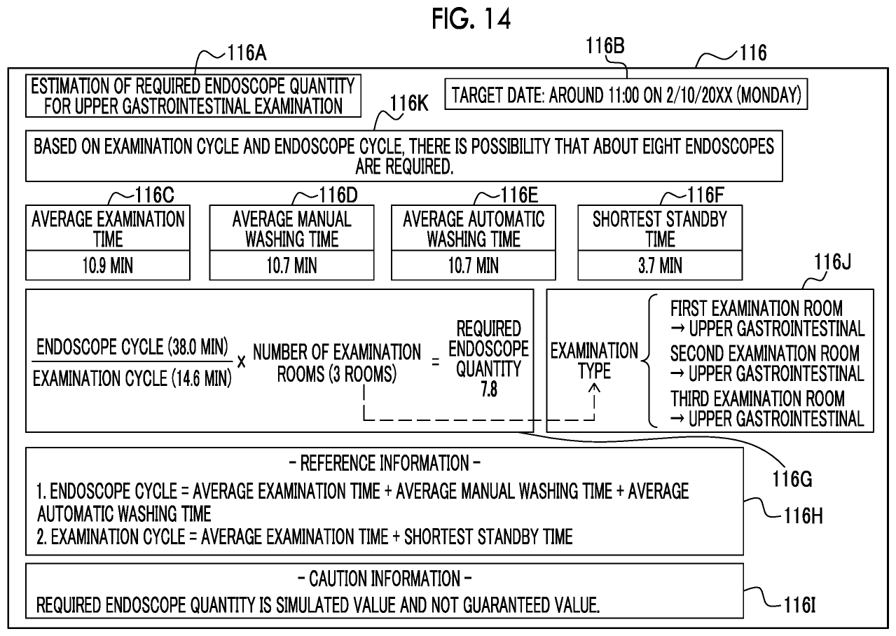

In this case, for example, the medical service support process shown in FIG. 13 is performed by the CPU 98. The flowchart shown in FIG. 14 is different from the flowchart shown in FIG. 11 in that it includes step ST206A to step ST220A instead of step ST206 to step ST220.

In step ST206A shown in FIG. 13, the specifying unit 98G specifies the peak operation date and time for each examination type with reference to the endoscopy management database 110 (see FIG. 9). A specification method of the examination type is the same as the specification method in step ST212A shown in FIG. 11.

In step ST208A, the derivation unit 98E calculates the endoscope cycle for each examination type for the peak operation date and time specified in step ST206A, with reference to the support device management database 113 (see FIG. 9).

In step ST210A, the derivation unit 98E calculates the examination cycle for each examination type for the peak operation date and time specified in step ST206A, with reference to the endoscopy management database 110 (see FIG. 9).

In step ST212A, the specifying unit 98G specifies the number of examination rooms for each examination type for the peak operation date and time specified in step ST206A.

In step ST214A, the derivation unit 98E calculates the required endoscope quantity for each examination type based on the endoscope cycle calculated in step ST208A, the examination cycle calculated in step ST210A, and the number of examination rooms specified in step ST212A.

In step ST216A, the generation unit 98F generates the notification information in which the required endoscope quantity for each examination type, which is calculated in step ST214A, is notified of.

In step ST218A, the generation unit 98F generates the medical service support information, which is obtained based on the required endoscope quantity calculated in step ST214A, for each examination type.

In step ST220A, the transmission unit 98B transmits the medical service support information, which is generated in step ST218A, for each examination type to the endoscope part management device main body 68 via the communication I/F 104 (see FIG. 7).

In a case where the medical service support information is transmitted for each examination type by executing the process of step ST220A shown in FIG. 13, in step ST110 shown in FIG. 10, the medical service support information is received for each examination type through the support device communication I/F 84 of the endoscope part management device 12. In step ST112 shown in FIG. 10, the display control unit 78D of the endoscope part management device 12 generates a medical service support screen 116 (see FIG. 14) for each examination type based on the medical service support information, and the generated medical service support screen 116 is displayed on the display 72 (see FIG. 14). In this case, for example, the medical service support screen 116 for each examination type is displayed on the display 72 in page units.

FIG. 14 shows an example of the medical service support screen 116 in a case where the type of endoscopy is upper gastrointestinal examination.

The medical service support screen 116 in the case of the upper gastrointestinal examination is merely an example, and there are also medical service support screens 116 related to other types of endoscopy. In a case where the display content of the medical service support screen 116 is switched to the display content related to another type of endoscopy, the display control unit 78D (see FIG. 4) switches the display content of the medical service support screen 116 to the display content related to another type of endoscopy, for example, in response to an instruction received by reception device 70 (see FIG. 1 and FIG. 3).

As an example, as shown in FIG. 14, on the medical service support screen 116, a screen title display field 116A, a required endoscope quantity calculation target date display field 116B, an average examination time display field 116C, an average manual washing time display field 116D, an average automatic washing time display field 116E, a shortest standby time display field 116F, a required endoscope quantity display field 116G, a reference information display field 116H, a caution information display field 116I, an examination type display field 116J, and an examination support comment display field 116K are displayed.

In the screen title display field 116A, a title that can enable recognition that the medical service support screen 116 is a screen displaying an estimate of the required endoscope quantity of a specific type of endoscopy (here, as an example, an upper gastrointestinal examination) is displayed. In the example shown in FIG. 14, “estimation of required endoscope quantity for upper gastrointestinal examination” is displayed in the screen title display field 116A.

In the required endoscope quantity calculation target date display field 116B, for the upper gastrointestinal examination, the peak operation date and time, which is specified in step ST206A shown in FIG. 13, is displayed.

In the average examination time display field 116C, for the upper gastrointestinal examination, the average examination time, which is used in the calculation of the endoscope cycle in step ST208A shown in FIG. 13 and in the calculation of the examination cycle in step ST210A shown in FIG. 13, is displayed. In the average manual washing time display field 116D, for the upper gastrointestinal examination, the average manual washing time, which is used in the calculation of the endoscope cycle in step ST208A shown in FIG. 13, is displayed. In the average automatic washing time display field 116E, for the upper gastrointestinal examination, the average automatic washing time, which is used in the calculation of the endoscope cycle in step ST208A shown in FIG. 13, is displayed. In the shortest standby time display field 116F, for the upper gastrointestinal examination, the shortest standby time, which is used in the calculation of the examination cycle in step ST210A shown in FIG. 13, is displayed.

In the required endoscope quantity display field 116G, for the upper gastrointestinal examination, the required endoscope quantity, which is calculated in step ST214A shown in FIG. 13, is displayed. Further, in the required endoscope quantity display field 116G, for the upper gastrointestinal examination, the expression used in the calculation of the required endoscope quantity, the endoscope cycle, the examination cycle, and the number of examination rooms are displayed.

In the reference information display field 116H, the reference information is displayed. In the example shown in FIG. 14, as an example of the reference information, an endoscope cycle calculation method for the upper gastrointestinal examination (in the example shown in FIG. 12, “endoscope cycle=average examination time+average manual washing time+average automatic washing time”) and an examination cycle calculation method for the upper gastrointestinal examination (in the example shown in FIG. 12, “examination cycle=average examination time+shortest standby time”) are shown.

In the caution information display field 116I, the caution information is displayed. In the example shown in FIG. 12, as an example of the caution information, a comment indicating that the required endoscope quantity, which is displayed in the required endoscope quantity display field 116G, for the upper gastrointestinal examination is a simulated value and not a guaranteed value is displayed.

In the examination type display field 116J, information indicating the type of endoscopy (here, as an example, an upper gastrointestinal examination), which is performed in each examination room 30 on the peak operation date and time, is displayed for each examination room 30.

In the examination support comment display field 116K, a comment related to the required endoscope quantity calculated based on the examination cycle for the upper gastrointestinal examination and on the endoscope cycle for the upper gastrointestinal examination is displayed.

In the example shown in FIG. 13, the required endoscope quantity is derived for each examination type based on the average examination time, the average manual washing time, the average automatic washing time, and the shortest standby time (see step ST214A shown in FIG. 13). The medical service support information based on the derived required endoscope quantity is transmitted to the endoscope part management device 12 for each examination type (see step ST220A shown in FIG. 13). Further, in the endoscope part management device 12, the medical service support screen 116 is generated based on the medical service support information and is displayed on the display 72. Since the medical service support screen 116 is presented to the user or the like in a state in which the medical service support information in accordance with the examination type is visualized, the user or the like can ascertain the required endoscope quantity in accordance with the examination type. Therefore, according to the present configuration, it is possible to support the efficient performance of the endoscope procedure service for each examination type. Further, since the medical service support information in accordance with the examination type is displayed on the medical service support screen 116, the user or the like can recognize the required endoscope quantity in accordance with the examination type.

In the embodiment described above, although an example of the embodiment has been described in which the medical service support information is generated based on the required endoscope quantity, the present disclosed technology is not limited to this. For example, the CPU 98 of the support management server 74 may acquire the current quantity of the endoscopes 24 and output information which includes information obtained based on the required endoscope quantity and the current endoscope quantity, as the medical service support information.

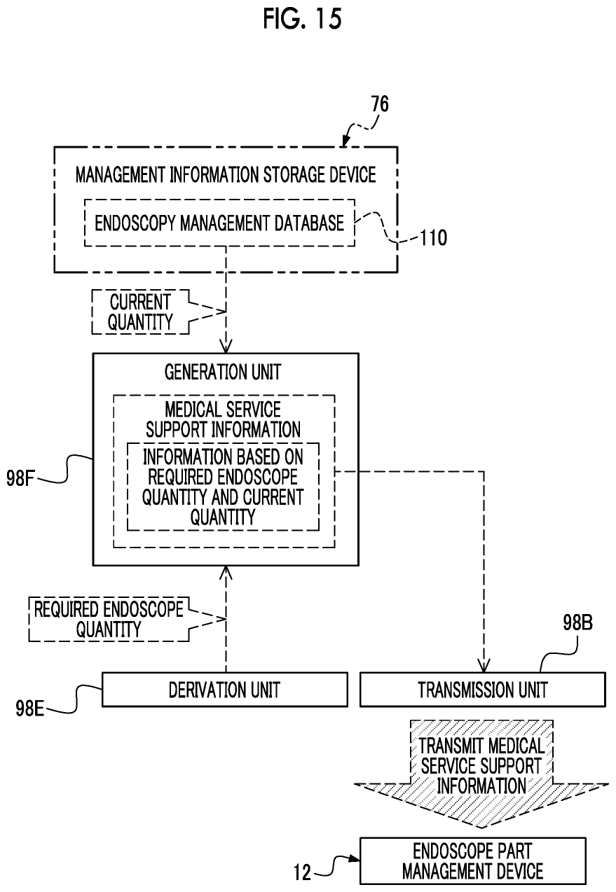

In this case, as shown in FIG. 15 as an example, the generation unit 98F acquires the current quantity of the endoscopes 24 based on the number of endoscope IDs included in the endoscopy information 44 (see FIG. 5) stored in the endoscopy management database 110. Here, although the number of endoscope IDs included in the endoscopy information 44 stored in the endoscopy management database 110 is regarded as the current quantity of the endoscopes 24, the present disclosed technology is not limited to this, and in practice, the quantity of endoscopes 24 that are actually owned by the medical installation facility may be used as the current quantity of the endoscopes 24.

The generation unit 98F acquires the required endoscope quantity from the derivation unit 98E. The generation unit 98F generates the medical service support information that includes information obtained based on the required endoscope quantity and the current endoscope quantity (hereinafter, also referred to as “first quantity related information”). The first quantity related information includes, for example, information indicating a difference degree between the required endoscope quantity and the current endoscope quantity (for example, a difference between the required endoscope quantity and the current endoscope quantity and/or the magnitude relationship between the required endoscope quantity and the current endoscope quantity).

The transmission unit 98B transmits the medical service support information including the first quantity related information to the endoscope part management device 12. In a case where the medical service support information including the first quantity related information is transmitted to the endoscope part management device 12, the medical service support information including the first quantity related information is received through the support device communication I/F 84 of the endoscope part management device 12. In step ST112 shown in FIG. 10, the display control unit 78D of the endoscope part management device 12 generates a medical service support screen with the first quantity related information based on the medical service support information including the first quantity related information, and the generated medical service support screen with the first quantity related information is displayed on the display 72.

The medical service support screen with the first quantity related information is different from the medical service support screens 114 and 116 in that the information which indicates the difference degree between the required endoscope quantity and the current endoscope quantity is displayed via a comment (for example, a comment expressing the difference degree between the required endoscope quantity and the current endoscope quantity), a figure (for example, a figure that can compare the required endoscope quantity and the current endoscope quantity), and/or a table (for example, a table that can compare the required endoscope quantity and the current endoscope quantity).

As described above, according to the example shown in FIG. 15, the CPU 98 of the support management server 74 acquires the current quantity of the endoscopes 24 and transmits the medical service support information, which includes the first quantity related information including information obtained based on the required endoscope quantity and the current endoscope quantity, to the endoscope part management device 12. The medical service support screen with the first quantity related information is displayed on the display 72. Therefore, the user or the like can ascertain the information obtained based on the required endoscope quantity and the current endoscope quantity. Moreover, since the information, which is based on the required endoscope quantity and the current endoscope quantity, is information indicating the difference degree between the required endoscope quantity and the current endoscope quantity, the user or the like can ascertain how much the required endoscope quantity deviates from the current endoscope quantity.

Further, the first quantity related information may be generated for each examination type and transmitted to the endoscope part management device 12 for each examination type, and the medical service support screen with the first quantity related information may be displayed on the display 72 for each examination type.

In the example shown in FIG. 15, although the current endoscope quantity is exemplified, the present disclosed technology is not limited to this. For example, the CPU 98 of the support management server 74 may acquire the quantity in which the endoscopes 24 are used within the default period and output information, which is obtained based on the required endoscope quantity and the quantity in which the endoscopes 24 are used within the default period, as the medical service support information.

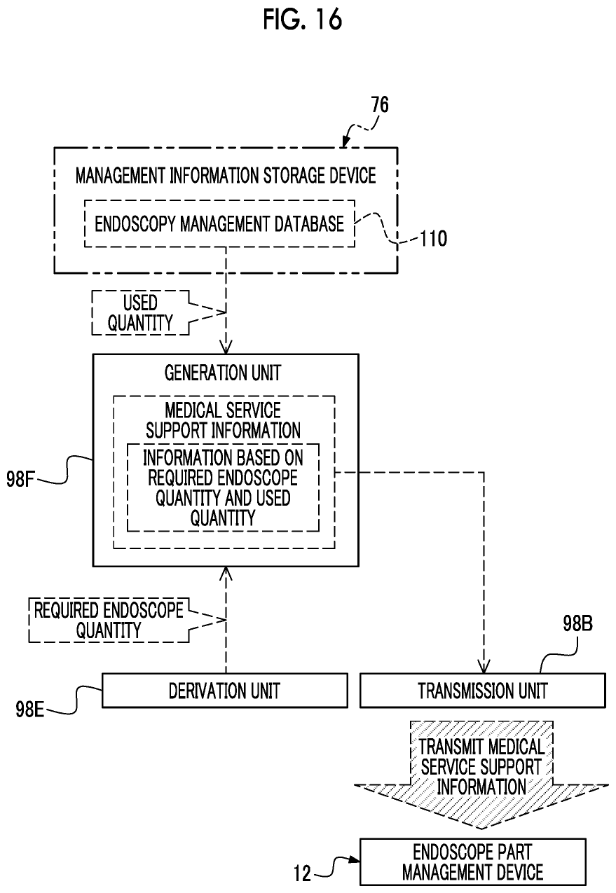

In this case, as shown in FIG. 16 as an example, the generation unit 98F acquires the used quantity of the endoscopes 24 with a record of being used within the default period (hereinafter, simply also referred to as “used endoscope quantity”) with reference to the endoscopy management database 110. Examples of the default period include the peak operation date and time. In addition to the peak operation date and time, the peak operation date may be used. Further, the default period refers to, for example, a period determined according to an instruction received by the reception device 70 (see FIG. 1 and FIG. 3). The default period is a period that can be defined in units of years, months, dates, days of the week, hours, minutes, and/or seconds, and may be a fixed period, may be an instruction given by a user or the like, or may be a variable period that is changed in a case where a specific condition (for example, a condition that the designated time points arrive) is satisfied.

The generation unit 98F acquires the required endoscope quantity from the derivation unit 98E. The generation unit 98F generates the medical service support information that includes information obtained based on the required endoscope quantity and the used endoscope quantity (hereinafter, also referred to as “second quantity related information”). The second quantity related information includes, for example, information indicating a difference degree between the required endoscope quantity and the used endoscope quantity (for example, a difference between the required endoscope quantity and the used endoscope quantity and/or the magnitude relationship between the required endoscope quantity and the used endoscope quantity).

The transmission unit 98B transmits the medical service support information including the second quantity related information to the endoscope part management device 12. In a case where the medical service support information including the second quantity related information is transmitted to the endoscope part management device 12, the medical service support information including the second quantity related information is received through the support device communication I/F 84 of the endoscope part management device 12. In step ST112 shown in FIG. 10, the display control unit 78D of the endoscope part management device 12 generates a medical service support screen with the second quantity related information based on the medical service support information including the second quantity related information, and the generated medical service support screen with the second quantity related information is displayed on the display 72.

The medical service support screen with the second quantity related information is different from the medical service support screens 114 and 116 in that the information which indicates the difference degree between the required endoscope quantity and the used endoscope quantity is displayed via a comment (for example, a comment expressing the difference degree between the required endoscope quantity and the used endoscope quantity), a figure (for example, a figure that can compare the required endoscope quantity and the used endoscope quantity), and/or a table (for example, a table that can compare the required endoscope quantity and the used endoscope quantity).

As described above, according to the example shown in FIG. 16, the CPU 98 of the support management server 74 acquires the used quantity of the endoscopes 24 and transmits the medical service support information, which includes the second quantity related information including information obtained based on the required endoscope quantity and the used endoscope quantity, to the endoscope part management device 12. The medical service support screen with the second quantity related information is displayed on the display 72. Therefore, the user or the like can ascertain the information obtained based on the required endoscope quantity and the used endoscope quantity. Moreover, since the information, which is obtained based on the required endoscope quantity and the used endoscope quantity, is information indicating the difference degree between the required endoscope quantity and the used endoscope quantity, the user or the like can ascertain how much the required endoscope quantity deviates from the used endoscope quantity.

Further, the second quantity related information may be generated for each examination type and transmitted to the endoscope part management device 12 for each examination type, and the medical service support screen with the second quantity related information may be displayed on the display 72 for each examination type.

In the embodiment described above, although an example of the embodiment has been described in which the generation unit 98F generates the medical service support information regardless of whether the endoscope 24 needs to be updated (for example, replaced), the present disclosed technology is not limited to this. For example, the types of endoscopes 24 may be categorized into a type in which an update is not required and a type in which an update is required, and the CPU 98 of the support management server 74 may output specification information capable of specifying the type in which the update is required based on an attribute of the endoscope 24.

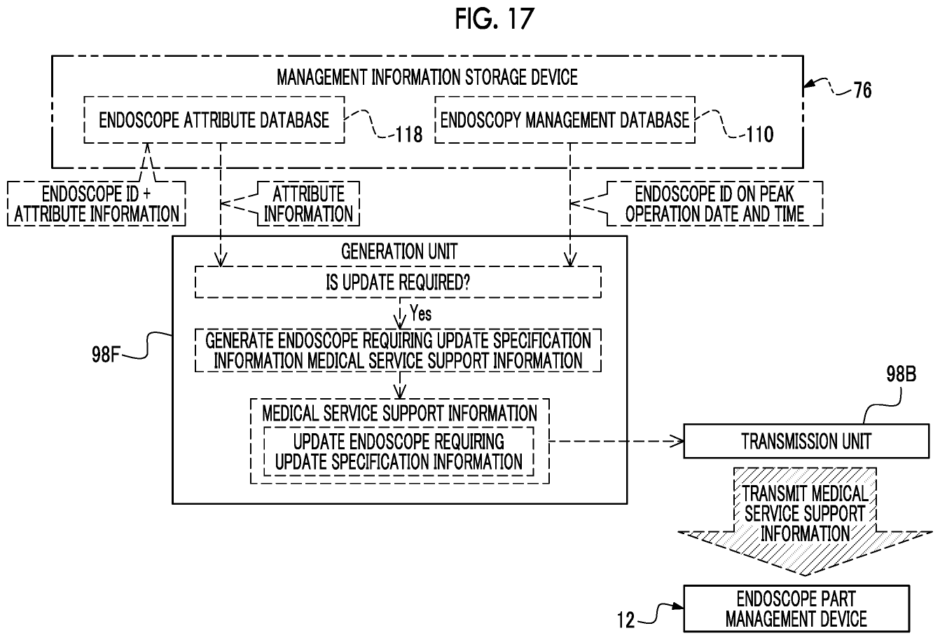

In this case, as shown in FIG. 17 as an example, in the management information storage device 76, a plurality of endoscope IDs, which are related to all the endoscopes 24 owned by the medical installation facility, and attribute information, which indicates the attributes of the endoscopes 24, are stored in an endoscope attribute database 118 in an associated manner.

Here, the attribute of the endoscope 24 refers to, for example, the cumulative number of times of use. The cumulative number of times of use is merely an example, and the attributes of the endoscope 24 may be cumulative operation time, frequency of use, repair history, the number of repairs, frequency of repairs, a failure rate, the repair amount, the purchase amount, and/or a medical treatment.

The generation unit 98F acquires the endoscope ID related to the endoscope 24 with a record of being used on the peak operation date and time from the endoscopy management database 110 and acquires the attribute information corresponding to the acquired endoscope ID from the endoscope attribute database 118.

The generation unit 98F determines whether or not the endoscope 24 which is specified based on the endoscope ID acquired from the endoscopy management database 110 is the endoscope 24 that requires an update with reference to the attribute information acquired from the endoscopy management database 110. For example, in a case where the attribute information is the cumulative number of times of use, it is determined that an update is required in a case where the cumulative number of times of use exceeds a threshold value (for example, 10,000 times). The threshold value to be compared with the cumulative number of times of use may be, for example, a value guaranteed by the manufacturer of the endoscope 24 or a value designated by the user or the like.

In a case where it is determined that the endoscope 24 which is specified based on the endoscope ID acquired from the endoscopy management database 110 is the endoscope 24 that requires an update, the generation unit 98F generates endoscope requiring update specification information that can specify the endoscope 24 that requires an update. The endoscope requiring update specification information includes the endoscope ID related to the endoscope 24 which is determined to require an update.

The generation unit 98F generates the medical service support information including the endoscope requiring update specification information. The transmission unit 98B transmits the medical service support information including the endoscope requiring update specification information to the endoscope part management device 12. In a case where the medical service support information including the endoscope requiring update specification information is transmitted to the endoscope part management device 12, the medical service support information including the endoscope requiring update specification information is received through the support device communication I/F 84 of the endoscope part management device 12. In step ST112 shown in FIG. 10, the display control unit 78D of the endoscope part management device 12 generates a medical service support screen with the endoscope requiring update specification information based on the medical service support information including the endoscope requiring update specification information, and the generated medical service support screen with the endoscope requiring update specification information is displayed on the display 72.

The medical service support screen with the endoscope requiring update specification information is different from the medical service support screens 114 and 116 in that information which can specify the endoscope 24 determined to require an update is displayed via a comment, a figure, and/or a table. Further, on the medical service support screen with the endoscope requiring update specification information, a comment or the like prompting the user or the like to update (for example, replace) the endoscope 24 may be displayed, or a screen introducing the latest endoscope 24 may be displayed on the display 72, in a state in which the endoscope 24 which is determined to require an update is associated with the specifiable information.

As described above, according to the example shown in FIG. 17, the CPU 98 of the support management server 74 transmits the endoscope requiring update specification information, which can specify the endoscope 24 that requires an update, to the endoscope part management device 12 based on the attribute information. The medical service support screen with the endoscope requiring update specification information is displayed on the display 72. Therefore, the user or the like can ascertain the endoscope 24 that is determined to require an update.

In the embodiment described above, although an example of the embodiment has been described in which the date on which an endoscopy is performed the highest number of times is defined as the peak operation date, the present disclosed technology is not limited to this. For example, the peak operation date may be a date on which the number of times the examination standby time (see FIG. 2) falls below the threshold value (for example, 3 minutes) is equal to or greater than a reference number of times. In this case, it is possible to specify the peak operation date even in a case where the number of times an endoscopy is performed cannot be ascertained. Note that the threshold value and the reference number of times may be fixed values or variable values that are changed by the user or the like. In other words, the operation degree of the medical device may be indicated by a value based on the number of times the examination standby time falls below the threshold value, and the reference degree may be indicated by the reference number of times. The number of times the examination standby time falls below the threshold value indicates the number of times the examination standby time for each predetermined period, such as for each day or for each hour, falls below the threshold value. Further, in a case where there are a plurality of medical devices that can be acquired by the medical service support device, the number of times the examination standby time falls below the threshold value indicates the total number of times the examination standby time falls below the threshold value in the plurality of medical devices. The threshold value may correspond to a preparation period required from the endoscopy end to the next endoscopy start in a medical installation facility, may be a fixed value, or may be a variable value set or changed by the user or the like. Further, the threshold value may be a variation value calculated based on the examination standby time of the endoscope procedure service performed in the past. For example, the average value of the examination standby time of the endoscope procedure service performed in the past, the minimum value of the examination standby time of the endoscope procedure service performed in the past, or a value obtained by adding or multiplying a predetermined value to the minimum value may be used. Further, the reference number of times corresponding to the reference degree is a default value. More specifically, it is a default value for determining a period during which endoscopy is intensively performed, such as the peak date or the peak time. The default value may be a fixed value set in advance, or a variable value changed according to a user input. Further, the default value may be a variation value calculated based on the number of times the examination standby time in the past falls below the threshold value. For example, the default value may be an average value of the number of times the examination standby time in the past falls below the threshold value or a variation value calculated based on the number of times the examination standby time in the past falls below the threshold value, for example, the maximum value of the number of times the examination standby time falls below the threshold value or a value obtained by adding or multiplying a predetermined value to the maximum value. Further, in a case where the operation degree of the medical device indicates the number of times the examination standby time falls below the threshold value for each first period (for example, one day), the highest value, which is within the number of times the examination standby time falls below the threshold value for each first period in a second period (for example, one week) longer than the first period, may be set as a default value.

In the embodiment described above, although the peak operation date is exemplified, the present disclosed technology is not limited to this, and the peak operation time period may be specified in a unit of time other than day, such as peak operation month or peak operation year.

In the embodiment described above, although the date and time on which the operation statuses of all the information processing apparatuses 36 peak is exemplified as the peak operation date and time, the present disclosed technology is not limited to this. For example, the date and time on which the operation statuses of all the endoscopes 24 peak may be defined as the peak operation date and time, or the date and time on which the operation statuses of all the washers 52 peak may be defined as the peak operation date and time.

In the embodiment described above, although the average washing time is exemplified as the standard washing time, the present disclosed technology is not limited to this, and a statistical value such as the median value, the most frequent value, the maximum value, the minimum value, or the percentile of the washing time for a predetermined period (for example, the peak operation date and time) may be used, or any time that is derived as the standard time required from when the endoscopy is ended to when washing of the endoscope 24 is ended may be used, instead of the average washing time.

In the embodiment described above, although the average manual washing time is exemplified as the first washing time, the present disclosed technology is not limited to this, and a statistical value such as the median value, the most frequent value, the maximum value, the minimum value, or the percentile of the manual washing time for a predetermined period (for example, the peak operation date and time) may be used, or any time that is derived as the standard time required for manual washing of the endoscope 24 may be used, instead of the average manual washing time.

In the embodiment described above, although the average automatic washing time is exemplified as the second washing time, the present disclosed technology is not limited to this, and a statistical value such as the median value, the most frequent value, the maximum value, the minimum value, or the percentile of the automatic washing time for a predetermined period (for example, the peak operation date and time) may be used, or any time that is derived as the standard time required for washing of the endoscope 24 by the washer 52 may be used, instead of the average automatic washing time.

In the embodiment described above, although the shortest standby time is exemplified as the standard standby time, the present disclosed technology is not limited to this, and a statistical value such as the median value, the most frequent value, the maximum value, the minimum value, or the percentile of the examination standby time for a predetermined period (for example, the peak operation date and time) may be used, or any time that is derived as the standard time required from when the N-th endoscopy is ended to when the (N+1)-th endoscopy is started may be used.

In the embodiment described above, although the average examination time is exemplified as the standard procedure service time, the present disclosed technology is not limited to this, and a statistical value such as the median value, the most frequent value, the maximum value, the minimum value, or the percentile of the examination time for a predetermined period (for example, the peak operation date and time) may be used, or any time that is derived as the standard time required from when the N-th endoscopy is started to when the N-th endoscopy is ended may be used.

In the embodiment described above, although an example of the embodiment has been described in which the operation of the information processing apparatus 36 is started by turning on the examination start button 38A, and the operation of the information processing apparatus 36 is stopped by turning off the examination start button 38A, the present disclosed technology is not limited to this. The operation of the information processing apparatus 36 may be started on a condition that the barcode 42 is read by the barcode reader 40, and the operation of the information processing apparatus 36 may be stopped on a condition that the barcode 42 is read again by the barcode reader 40. The same applies to the start and stop of the operation of the washer 52.

An example has been described in which the start operation and the stop operation of the information processing apparatus 36 are executed by pressing down the examination start button 38A, but this is merely an example. The examination start button 38A is not limited to a physical button. The operation of the information processing apparatus 36 may be started or the operation of the information processing apparatus 36 may be stopped by detecting nearness of at least one softkey displayed on a touch panel, such as a touch panel display. Further, the start operation and the stop operation of the information processing apparatus 36 may be controlled by a voice input and/or a gesture input. Further, a noncontact human sensor may be provided in a default region of the examination room 30 or the information processing apparatus 36, and the operation of the information processing apparatus 36 may be started or the operation of the information processing apparatus 36 may be stopped by causing the noncontact human sensor to detect a human body. The same applies to the control of the start operation and the stop operation by using the washing start button 60A of the washer 52.

In the embodiment described above, although an example of the embodiment has been described in which the medical service support information is generated based on the required endoscope quantity as a target of the endoscope part, the present disclosed technology is not limited to this, and the medical service support information may be generated based on the required endoscope quantity in a case where an endoscopy is performed in units of clinical departments (for example, internal medicine or surgery) or medical institutions (for example, clinic or general hospital).

In the embodiment described above, although the examination room 30 has been exemplified as an example, it does not necessarily have to be one room, and may be a space obtained by dividing one room by a curtain and/or a stand or the like.

In the above embodiment, although the endoscope processor device 32 and the information processing apparatus 36 are separate units, the present disclosed technology is not limited to this, and the endoscope processor device 32 and the information processing apparatus 36 may be integrated. In this case, for example, a device corresponding to the information processing apparatus 36 may be incorporated into the endoscope processor device 32, or a device corresponding to the endoscope processor device 32 may be incorporated into the information processing apparatus main body 38.

In the embodiment described above, although an example of the embodiment has been described in which the medical service support information is visualized and presented to the user or the like by displaying the medical service support screens 114 and 116 on the display 72, the present disclosed technology is not limited to this. For example, the medical service support information may be audibly output by a sound reproducing device, or the medical service support information may be printed on a recording medium (for example, paper) by a printer and output, instead of or together with the visual presentation by the display 72 described above.

In the embodiment described above, although the endoscope handling service including an endoscopy, which is an example of the “endoscope procedure service” according to the present disclosed technology, has been described, the present disclosed technology is not limited to this, and a medical device handling service including a medical device procedure service other than an endoscopy may be used. For example, the endoscope procedure service may be a service including endoscopic surgery and/or endoscopic treatment. Examples of endoscopic surgery include laparoscopic surgery and/or treatment, thoracoscopic surgery and/or treatment, cystoscopic surgery and/or treatment, choledoscopic surgery and/or treatment, spinal endoscopy and/or therapy, angioscopy surgery and/or treatment, and epidural endoscopic surgery and/or treatment.

In the embodiment described above, although the endoscope 24, the information processing apparatus 36, and the washer 52 are described as examples of the medical device, the present disclosed technology is not limited to this, and the medical device may be a medical device other than the endoscope 24, the information processing apparatus 36, and the washer 52. Examples of the medical device other than the endoscope 24, the information processing apparatus 36, and the washer 52 include a medical management device (for example, a device having a processor and a memory) corresponding to the information processing apparatus 36, a medical accessory device that is attachably and detachably connected to a medical management device in the same way that the endoscope 24 is attachably and detachably connected to the information processing apparatus 36 (for example, replaceable ultrasonic probes and/or replaceable therapeutic tools used in dental treatment, or the like), and a washer that washes the medical accessory device similar to the washer 52 that washes the endoscope 24.

In the embodiment described above, although the barcode 42 and the subject barcode are exemplified, another two-dimensional code such as a quick response (QR) code (registered trademark) may be used, or a noncontact storage medium using radio frequency identification (RFID) technology may be used, instead of at least one of the barcode 42 or the subject barcode. Further, in this case, the endoscope 24 or a device that can acquire information which is capable of specifying a subject may be applied by recognizing these two-dimensional codes and/or noncontact storage media instead of the barcode readers 40 and 64.

In the embodiment described above, although the medical service support device 20 is exemplified, a computer, which is used together with the endoscope part management device 12 on-premises, may be caused to execute the medical service support processing program 108 (see FIG. 8) instead of the medical service support device 20. Further, the management information storage device 76 may also be used on-premises together with the endoscope part management device 12.

Further, in the embodiment described above, although an example of the embodiment has been described in which the medical service support processing program 108 is stored in the storage 100, the medical service support processing program 108 may be stored in any portable storage medium such as an SSD or universal serial bus (USB) memory, or the medical service support processing program 108 may be stored in a non-temporary storage medium. The medical service support processing program 108 stored in the non-temporary storage medium is installed, for example, in the support management server 74 or the like.

Further, in the embodiment described above, although the support management server 74 including the CPU 98, the storage 100, and the memory 102 has been exemplified, the present disclosed technology is not limited to this, and a device including an application specific integrated circuit (ASIC), a field-programmable gate array (FPGA), and/or a programmable logic device (PLD) may be applied instead of the support management server 74 or together with the support management server 74.

As a hardware resource for executing the medical service support process described in the above embodiment, the following various processors can be used. Examples of the processor include a CPU, which is a general-purpose processor that functions as a hardware resource for executing the medical service support process by executing software, that is, a program. Further, examples of the processor include a dedicated electric circuit, which is a processor having a circuit configuration specially designed for executing specific processing such as an FPGA, a PLD, or an ASIC. Any processor has a memory built in or connected to it, and any processor uses the memory to execute a terminal side process.

The hardware resource for executing the medical service support process may be configured with one of these various processors or may be configured with a combination (for example, a combination of a plurality of FPGAs or a combination of a CPU and an FPGA) of two or more processors of the same type or different types. Further, the hardware resource for executing the medical service support process may be one processor.

As an example of a configuration with one processor, first, one processor is configured with a combination of one or more CPUs and software, and there is an embodiment in which this processor functions as a hardware resource for executing the medical service support process. Secondly, as typified by a system-on-a-chip (SoC), there is an embodiment in which a processor that implements the functions of the entire system including a plurality of hardware resources for executing the medical service support process with one IC chip is used. As described above, the medical service support process is implemented by using one or more of the above-mentioned various processors as a hardware resource.

Further, as the hardware-like structure of these various processors, more specifically, an electric circuit in which circuit elements such as semiconductor elements are combined can be used. Further, the above-mentioned terminal side process is merely an example. Therefore, it goes without saying that unnecessary steps may be deleted, new steps may be added, or the processing order may be changed within a range that does not deviate from the purpose.

The contents described above and the contents shown in the illustrations are detailed explanations of the parts related to the present disclosed technology and are merely an example of the present disclosed technology. For example, the description related to the configuration, function, action, and effect described above is an example related to the configuration, function, action, and effect of a portion according to the present disclosed technology. Therefore, it goes without saying that unnecessary parts may be deleted, new elements may be added, or replacements may be made to the contents described above and the contents shown in the illustrations, within the range that does not deviate from the purpose of the present disclosed technology. Further, in order to avoid complications and facilitate understanding of the parts of the present disclosed technology, in the contents described above and the contents shown in the illustrations, the descriptions related to common technical knowledge or the like that do not require special explanation in order to enable the implementation of the present disclosed technology are omitted.

In the present specification, “A and/or B” is synonymous with “at least one of A or B”. That is, “A and/or B” means that it may be only A, it may be only B, or it may be a combination of A and B. Further, in the present specification, in a case where three or more matters are connected and expressed by “and/or”, the same concept as “A and/or B” is applied.

All documents, patent applications, and technical standards described in the present specification are incorporated in the present specification by reference to the same extent in a case where it is specifically and individually described that the individual documents, the patent applications, and the technical standards are incorporated by reference.

What is claimed is:

1. A medical service support device comprising:
a processor; and
a memory that is connected to or built into the processor,
wherein the processor is configured to:
derive a required quantity of endoscopes based on, in a case where N is a natural number, standard procedure service time, from when a first endoscope procedure service, which is an N-th endoscope procedure service performed by an information processing apparatus used together with the endoscope, is started to when the first endoscope procedure service is ended, standard standby time, from when the first endoscope procedure service is ended to when a second endoscope procedure service, which is an (N+1)-th endoscope procedure service performed by the information processing apparatus, is started, and standard washing time, from when the first endoscope procedure service is ended to when washing of the endoscope is ended; and
output medical service support information obtained based on the required quantity.

2. The medical service support device according to claim 1,
wherein the processor is configured to:
acquire the standard procedure service time, the standard standby time, and the standard washing time according to type of the endoscope procedure service;
derive the required quantity according to the type based on the standard procedure service time, the standard standby time, and the standard washing time; and
output the medical service support information according to the type.

3. The medical service support device according to claim 1,
wherein the processor is configured to derive the required quantity based on the number of medical sites where the endoscope procedure service is performed, the standard procedure service time, the standard standby time, and the standard washing time.

4. The medical service support device according to claim 1,
wherein the processor is configured to derive the required quantity based on a ratio of time, which includes the standard procedure service time and the standard washing time, with respect to time, which includes the standard procedure service time and the standard standby time.

5. The medical service support device according to claim 1,
wherein the standard washing time is time that includes first washing time derived as standard time required for manual washing of the endoscope and second washing time derived as standard time required for washing of the endoscope by a washer.

6. The medical service support device according to claim 1,
wherein the medical service support information is presented by a presentation device.

7. The medical service support device according to claim 1,
wherein the medical service support information is information that includes notification information in which the required quantity is notified of.

8. The medical service support device according to claim 1,
wherein the processor is configured to:
acquire a current quantity of the endoscopes; and
output information which includes information obtained based on the required quantity and the current quantity, as the medical service support information.

9. The medical service support device according to claim 8,
wherein the information, which is obtained based on the required quantity and the current quantity, is information indicating a difference degree between the required quantity and the current quantity.

10. The medical service support device according to claim 1,
wherein the processor is configured to:
acquire a used quantity of the endoscopes within a default period; and
output information which includes information obtained based on the required quantity and the used quantity, as the medical service support information.

11. The medical service support device according to claim 1,
wherein types of the endoscope are categorized into a type in which an update is not required and a type in which an update is required, and
the processor is configured to output information which includes specification information capable of specifying the type in which the update is required, as the medical service support information, based on the required quantity and an attribute of the endoscope.

12. The medical service support device according to claim 1,
wherein the medical service support information is presented by a presentation device according to type of the endoscope procedure service.

13. The medical service support device according to claim 1,
wherein the processor is configured to derive the required quantity for a time period when an operation degree of a medical device, which is used in an endoscope handling service including the endoscope procedure service, is equal to or higher than a reference degree.

14. The medical service support device according to claim 13,
wherein the time period when the operation degree of the medical device is equal to or higher than the reference degree is a time period when a value based on the number of endoscope procedure services is equal to or greater than a reference value.

15. The medical service support device according to claim 13,
wherein the time period when the operation degree of the medical device is equal to or higher than the reference degree is a time period when the number of times standby time of the endoscope procedure service falls below a threshold value is equal to or greater than a reference number of times.

16. A medical service support device comprising:
a processor; and
a memory that is connected to or built into the processor,
wherein the processor is configured to:
derive a required quantity of medical accessory devices based on, in a case where N is a natural number, standard procedure service time, from when a first medical procedure service, which is an N-th medical procedure service performed by an information processing apparatus, is started to when the first medical procedure service is ended, standard standby time, from when the first medical procedure service is ended to when a second medical procedure service, which is an (N+1)-th medical procedure service performed by the information processing apparatus, is started, and standard washing time, from when the first medical procedure service is ended to when washing of the medical accessory device, which is attachably and detachably connected to the information processing apparatus, is ended; and
output medical service support information obtained based on the required quantity.

17. A medical service support method comprising:
deriving a required quantity of endoscopes based on, in a case where N is a natural number, standard procedure service time, from when a first endoscope procedure service, which is an N-th endoscope procedure service performed by an information processing apparatus used together with the endoscope, is started to when the first endoscope procedure service is ended, standard standby time, from when the first endoscope procedure service is ended to when a second endoscope procedure service, which is an (N+1)-th endoscope procedure service performed by the information processing apparatus, is started, and standard washing time, from when the first endoscope procedure service is ended to when washing of the endoscope is ended; and
outputting medical service support information obtained based on the required quantity.

18. A non-transitory computer-readable storage medium storing a program executable by a computer to perform a process comprising:
deriving a required quantity of endoscopes based on, in a case where N is a natural number, standard procedure service time, from when a first endoscope procedure service, which is an N-th endoscope procedure service performed by an information processing apparatus used together with the endoscope, is started to when the first endoscope procedure service is ended, standard standby time, from when the first endoscope procedure service is ended to when a second endoscope procedure service, which is an (N+1)-th endoscope procedure service performed by the information processing apparatus, is started, and standard washing time, from when the first endoscope procedure service is ended to when washing of the endoscope is ended; and
outputting medical service support information obtained based on the required quantity.
